(19) United States
(12) Patent Application Publication
(10) Pub. No.: US 2026/0027041 A1
(43) Pub. Date: Jan. 29, 2026

(54) Formulations and Methods for a GLP-1 Agonist Microneedle Patch

(71) Applicant: Micron Biomedical, Inc., Atlanta, GA (US)

(72) Inventors: Devin V. McAllister, Marietta, GA (US); Marly I. Richter-Roche, Atlanta, GA (US)

(21) Appl. No.: 19/284,639

(22) Filed: Jul. 29, 2025

Related U.S. Application Data
(60) Provisional application No. 63/676,750, filed on Jul. 29, 2024.

(51) Int. Cl. A61K 9/00; A61K 38/26; A61K 47/32; A61K 47/36

(52) U.S. Cl. CPC A61K 9/0021; A61K 38/26; A61K 47/32; A61K 47/36

(57) ABSTRACT

Microneedles are provided that includes a microneedle body including a tip end portion configured for insertion into a biological tissue, wherein at least the tip end portion includes a mixture of a GLP-1 agonist and an excipient composition which comprises a carbohydrate and a polymer, wherein the mass ratio of the carbohydrate to the polymer in the excipient composition is at least 1.5:1. A microneedle patch is provided that includes a base, and an array of such microneedles extending from the base.

### CROSS REFERENCE TO RELATED APPLICATIONS

This application claims priority to U.S. Provisional Patent Application No. 63/676,750 filed Jul. 29, 2024, which is incorporated herein by reference.

### BACKGROUND

The present application is generally in the field of microneedle patches for the administration of bioactive agents to humans, and more specifically, is in the field of microneedle patches for delivery of a Glucagon-Like Peptide-1 (GLP-1) agonist.

GLP-1 receptor agonists are a class of medications that mimic the action of the natural GLP-1 hormone, which plays a key role in regulating blood sugar, appetite, and digestion. These drugs stimulate insulin secretion, suppress glucagon release, and slow gastric emptying, making them highly effective for managing type 2 diabetes and promoting weight loss. Popular GLP-1 agonists include semaglutide (Ozempic, Wegovy), liraglutide (Victoza, Saxenda), and tirzepatide (Mounjaro, Zepbound), with newer agents like CagriSema and orforglipron in development. Their dual benefits in glycemic control and weight management have made them a cornerstone of modern metabolic therapy. The GLP-1 agonist landscape is rapidly evolving, with expanded indications being explored for conditions such as chronic kidney disease, heart failure, and even certain cancers. However, GLP-1 agonists require frequent administration via injection, generally once per day to once per week and can be associated with pain or discomfort. Thus, there remains a need for the development of other and more convenient administration methods.

Microneedle arrays represent a cutting-edge technology in drug and vaccine delivery. This drug delivery platform is made from biocompatible materials, which may dissolve upon penetrating the skin, and release their payload directly into a patient's body. The platform may offer several advantages over traditional injections, including thermostability, reduced pain, improved patient compliance, ease of use, elimination of hypodermic needles, and in some cases, the elimination of sharps waste.

### BRIEF SUMMARY

In one aspect, a microneedle is provided that includes a microneedle body including a tip end portion configured for insertion into a biological tissue, wherein at least the tip end portion includes a mixture of a GLP-1 agonist and an excipient composition which comprises a carbohydrate and a polymer. The mass ratio of the carbohydrate to the polymer in the excipient composition can be at least 1.5:1. In some preferred embodiments, the carbohydrate is a sugar and the polymer is a hydrophilic polymer. For example, the GLP-1 agonist may comprise semaglutide, the polymer may comprise PVA, and the carbohydrate may comprise dextrose. In some preferred embodiments, the GLP-1 agonist in the excipient composition may have a thermostability of at least two weeks at 40° C. In another aspect, a microneedle patch is provided that includes a base, and an array of any of the foregoing GLP-1 agonist-containing microneedles extending from the base.

In still another aspect, kits are provided that include a plurality of microneedle patches comprising the GLP-1 agonist and the excipient composition; and instructions for using the microneedle patches to treat a subject, wherein the instructions comprise dosing frequency and/or directions for applying the microneedles of the microneedle patches to the subject's skin or other biological tissue. The microneedle patches of the kit may contain different doses of the GLP-1 agonist. Each dose may contain, for example, from 0.01 to 1.5 mg of the GLP-1 agonist. Each microneedle patch contains an array of microneedles and the microneedle array may contain, for example, about 0.25 mg to 2.5 mg of the GLP-1 agonist per cm² of the array.

In yet another aspect, methods are provided for administering a GLP-1 agonist to a subject comprising applying to a tissue of the subject, e.g., the subject's skin, at least one of the disclosed microneedle patches in a manner to cause the tip portions of the microneedles to be inserted into the tissue and to thereby release the GLP-1 agonist into the tissue. The applying preferably is effective to deliver at least 0.05 milligrams of the GLP-1 agonist from the microneedle patch, preferably with a delivery efficiency of the GLP-1 agonist from the microneedle patch which is at least 60%, more preferably from 85% to 95%. The methods may be effective to administer a pharmaceutically acceptable amount of the GLP-1 agonist to the subject to treat diabetes, obesity, or another metabolic condition in the subject, or to aid in weight loss in the subject or to treat an addiction, kidney disease, and/or cardiovascular disease in the subject.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments. Elements and/or components are not necessarily drawn to scale.

FIG. 1 is a perspective view of a microneedle array according to one or more embodiments of the present disclosure.

FIG. 2 is a process flow diagram illustrating a process for making a microneedle array according to one or more embodiments of the present disclosure.

FIG. 3 is a chromatogram demonstrating peak SG elution from a sample formulation during liquid chromatography, according to one or more embodiments of the present disclosure.

FIG. 4 is a graph of the calibration curve for formulations of SG, according to one or more embodiments of the present disclosure.

FIG. 5 is a graph of the SG content of liquid formulations and reconstituted films, according to one or more embodiments of the present disclosure.

FIGS. 6A-6C are chromatograms demonstrating peak SG elution during liquid chromatography for formulations of FIG. 5, according to one or more embodiments of the present disclosure.

FIG. 7 is a graph of the stability of SG formulations having different excipient compositions over time, according to one or more embodiments of the present disclosure.

FIG. 8 is a chromatogram demonstrating peak SG elution during liquid chromatography for formulations of FIG. 7, according to one or more embodiments of the present disclosure.

FIG. 9 is a graph of the stability of SG formulations having polymer and sugar excipient compositions, according to one or more embodiments of the present disclosure.

FIG. 10 is a graph of the stability of SG formulations having different excipient to active ratios, according to one or more embodiments of the present disclosure.

FIG. 11 is a graph of the stability of dissolvable microneedle patches formulated with SG, PVA and glucose, according to one or more embodiments of the present disclosure.

FIG. 12 is a chromatogram demonstrating peak SG elution during liquid chromatography for the dissolvable microneedle patches of FIG. 11, according to one or more embodiments of the present disclosure.

FIG. 13 is a chromatogram demonstrating peak SG elution during liquid chromatography for microneedle patches, according to one or more embodiments of the present disclosure.

FIG. 14 is a graph comparing the SG content of microneedle patches with sugar and polymer excipient compositions, according to one or more embodiments of the present disclosure.

FIG. 15 is a graph comparing the SG content of microneedle patches with sugar and polymer excipient compositions, according to one or more embodiments of the present disclosure.

FIG. 16 is a graph comparing the SG content of microneedle patches containing an excipient compositions and buffer, according to one or more embodiments of the present disclosure.

FIG. 17 is a graph comparing the SG content of microneedle patches containing an excipient composition and buffer, according to one or more embodiments of the present disclosure.

FIG. 18 is a graph comparing the SG content of microneedle patches prepared with a excipient compositions and buffers, according to one or more embodiments of the present disclosure.

FIG. 19 is a graph of the stability of microneedle patches formulated with SG and varying excipient to active ratios, according to one or more embodiments of the present disclosure.

FIG. 20 is a graph comparing the SG content of microneedle patches having different excipient compositions and excipient to active ratios, according to one or more embodiments of the present disclosure.

FIG. 21 is a graph comparing the SG content of microneedle patches having different excipient compositions following terminal sterilization, according to one or more embodiments of the present disclosure.

FIG. 22 is a graph comparing the SG content of microneedle patches having different excipient compositions following terminal sterilization and storage, according to one or more embodiments of the present disclosure.

FIG. 23 is a graph comparing the SG content of microneedle patches after terminal sterilization and storage, according to one or more embodiments of the present disclosure.

FIG. 24 is a chromatogram demonstrating peak SG elution for terminally sterilized microneedle patches having different excipient compositions, according to one or more embodiments of the present disclosure.

FIG. 25 is a graph comparing the SG content of microneedle patches having excipient compositions with varying polymer to sugar ratios and buffers, according to one or more embodiments of the present disclosure.

### DETAILED DESCRIPTION

Drug delivery systems are disclosed for administering a GLP-1 agonist to a subject, such as a human patient in need thereof. In a particular embodiment, the drug delivery system includes microneedles, such as an array of microneedles, which may be in the form of a patch comprising a base and an array of the microneedles. The microneedles of the patch are configured to penetrate a biological tissue, such as the subject's skin, and release a dose of a GLP-1 agonist. In a particular preferred embodiment, the patch comprises microneedles that dissolve following insertion into the subject's tissue to release the GLP-1 agonist—such a patch may be referred to herein as GLP-1 dMP (dissolvable Microneedle Patch). The dissolution may be sufficient to separate the GLP-1-agonist containing microneedle from a base, or to separate at least the distal tip end portion of the microneedle from a proximal portion of the microneedle. In some embodiments, the GLP-1 agonist is dulaglutide, exenatide, liraglutide, lixisenatide, semaglutide, semaglutide analogues, tirzepatide, or a combination thereof.

The presently disclosed microneedles and arrays advantageously may comprise commercially scalable formulations to produce dissolvable microneedle patches containing a GLP-1 agonist, as well as improved methods to treat subjects with the GLP-1 agonists. The GLP-1 agonist microneedle patches provided herein enable skin penetration, rapid dissolution in the skin, and sufficient delivery of the GLP-1 agonist and preserve the potency of the GLP-1 agonist in the microneedle. This may be achieved through delivery efficiency, diameter of the microneedle array, number of microneedles within the array, excipient composition, stability of the GLP-1 agonist within the microneedles, and/or the ability to withstand terminal sterilization.

### Overview

In some preferred embodiments, a microneedle is provided that has a tip end portion that is configured for insertion into skin or another biological tissue, wherein at least the tip end portion includes a mixture of a GLP-1 agonist and an excipient composition which comprises a carbohydrate and a polymer, preferably wherein the mass ratio of the carbohydrate to the polymer in the excipient composition is at least 1.5:1. The carbohydrate may be a sugar or sugar alcohol. In some embodiments, the carbohydrate includes or consists of sucrose, glucose, maltose, dextrose, trehalose, or a combination thereof. In some embodiments, the polymer includes or consists of a hydrophilic polymer, such as a polylactic acid (PLA), polyglycolic acid (PGA), polyvinyl pyrrolidone (PVP), and/or polyethylene glycol (PEG). In preferred embodiments, the polymer has a molecular weight between 5,000 and 60,000 Daltons. The excipient composition optionally may include one or more additional pharmaceutically acceptable excipients. In some embodiments, the GLP-1 agonist is semaglutide, tirzepatide, exenatide, or liraglutide.

The mass ratio of the GLP-1 agonist to the excipient composition in the microneedle preferably provides a commercially relevant dose of a GLP-1 agonist to be formulated in the microneedle. In some embodiments, the mass ratio of the GLP-1 agonist to the excipient composition, e.g., in the tip end portion of the microneedle, is from about 0.5:1 to about 2:1. In some embodiments, the mass ratio of the carbohydrate to the polymer in the excipient composition ranges from 1.5:1 to 10:1, for example between 1.5:1 and 8:1, and may preferably range from 2:1 to 5:1 (carbohydrate: polymer). The relatively higher proportion of carbohydrate (e.g., sugar) to polymer in the microneedle has been found to be advantageous for GLP-1 dMPs.

In some specific embodiments, the mass ratio of the GLP-1 agonist to the excipient composition in the mixture forming the tip end portion of the microneedle is about 1:1. In some specific embodiments, the GLP-1 agonist comprises semaglutide, the polymer comprises PVA, and the carbohydrate comprises dextrose. In some cases, the mass ratio of dextrose to PVA in the excipient composition is between 1.5:1 and 4.5:1, preferably 4:1. In some cases, the excipient composition comprises PVA, dextrose, and phosphate buffer.

In some embodiments, the excipient composition in the mixture forming the tip end portion of the microneedle is about 60% by weight dextrose and about 40% by weight PVA. In some embodiments, the excipient composition in the mixture forming the tip end portion of the microneedle is about 70% by weight dextrose and about 30% by weight PVA. In some embodiments, the excipient composition in the mixture forming the tip end portion of the microneedle is about 80% by weight dextrose and about 20% by weight PVA.

In some embodiments, the excipient composition in the mixture forming the tip end portion of the microneedle comprises PVA and maltose. In some embodiments, the excipient composition in the mixture forming the tip end portion of the microneedle comprises PVP, dextrose, and glycerol. In some embodiments, the excipient composition in the mixture forming the tip end portion of the microneedle comprises PVA and glucose, for example, 70% by weight glucose and 30% by weight PVA.

In some embodiments, the microneedle patch comprises a microneedle array which contains at least, or about, or exactly, 0.01 mg, 0.05 mg, 0.0625 mg, 0.125 mg, 0.25 mg, 0.5 mg, 1.0 mg, 1.5 mg, 2.0 mg, or 5.0 mg of a GLP-1 agonist. These amounts may be a full dose or a fractional dose of the GLP-1 agonist. In a particular embodiment, the microneedle patch contains and is configured to administer at least 1 mg of a GLP-1 agonist, such as semaglutide, to a subject.

The microneedle arrays preferably are configured to provide both the appropriate dosage of a GLP-1 agonist to be administered and ease of application by a subject. In certain embodiments, the microneedle arrays may contain at least 160 microneedles comprising the GLP-1 agonist. In some embodiments, the array and the microneedle patch may have from 100 microneedles to 500 microneedles, e.g., from 160 to 360 microneedles. Each microneedle, or at least its tip end portion, may be conical in shape and have length that is at least, about, or exactly, 300 μm, 400 μm, 500 μm, 600 μm, 700 μm, 800 μm, or 900 μm.

The microneedles of the array may be arranged and oriented into various array shapes, i.e., the shape of the perimeter of the array. Preferred shapes may facilitate uniform insertion of the microneedles into the biological tissue upon manual application. In some embodiments, the microneedles of the array are arranged in a circular or substantially circular pattern. For example, the microneedles may be positioned to define a circular perimeter of the array viewed in an axial direction of the microneedles, the perimeter having diameter of from about 0.5 cm to about 2 cm, e.g., 1 cm, 1.5 cm, or 2 cm. In a particular embodiment, the perimeter of the array has diameter of 0.5 cm and the array of microneedles comprises about 0.05 mg of the GLP-1 agonist. In another particular embodiment, the perimeter of the array has diameter of 1 cm and the array of microneedles comprises about 1.25 mg of the GLP-1 agonist.

In a particularly preferred embodiment, the microneedle array contains about 1.25 mg of a GLP-1 agonist, has about 160 microneedles that are about 700 μm in length, and has a substantially circular perimeter shape that has a diameter of about 1 cm. In a specific embodiment, the GLP-1 agonist of this array is semaglutide.

Advantageously and preferably, the GLP-1 agonist-containing microneedles and microneedle arrays are thermostable (i.e., retain microneedle functionality (able to penetrate skin and readily dissolve in the skin) and potency of the GLP-1 agonist upon short or prolonged exposure to elevated temperatures). In some embodiments, the thermostability is at least two weeks at 40° C. In other embodiments, the thermostability is at least 12 months at 5° C. In further embodiments, the thermostability is at least 1, 2, or 3 months at 40° C. In other embodiments, the thermostability is at least 6, 12, 24, or 36 months at 5° C. In other embodiments, the thermostability is at least 12, 24, or 36 months at ambient conditions for all climate zones (i.e., Zone 1-Temperate (21+/−2° C.), Zone 2-Mediterranean/Subtropical (25+/−2° C.), Zone 3-Hot/Dry (30+/−2° C.), and Zone 4-Hot/Humid/Tropical (30 +/−2° C., 65 or 75% RH) as defined by ICH guidelines.

In preferred embodiments, the microneedles, microneedle arrays, and microneedle patches described herein are provided in a sterilized form, at least prior to human use. In preferred embodiments, the physical and mechanical properties of the microneedles are maintained following sterilization. In one embodiment, terminal sterilization is used to sterilize the microneedle patch. Terminal sterilization refers to the process of sterilizing the final, packaged microneedle product to ensure it is free from viable microorganisms, or meets a predetermined sterility assurance level, before use to ensure patient safety. In some embodiments, terminal sterilization of the microneedle products provided herein are accomplished by, for example, irradiation by gamma, X-ray, or E-beam.

In alternate embodiments, the microneedles, microneedle arrays, and microneedle patches described herein are provided non-sterile but with a low bioburden specification that is suitable for their intended use.

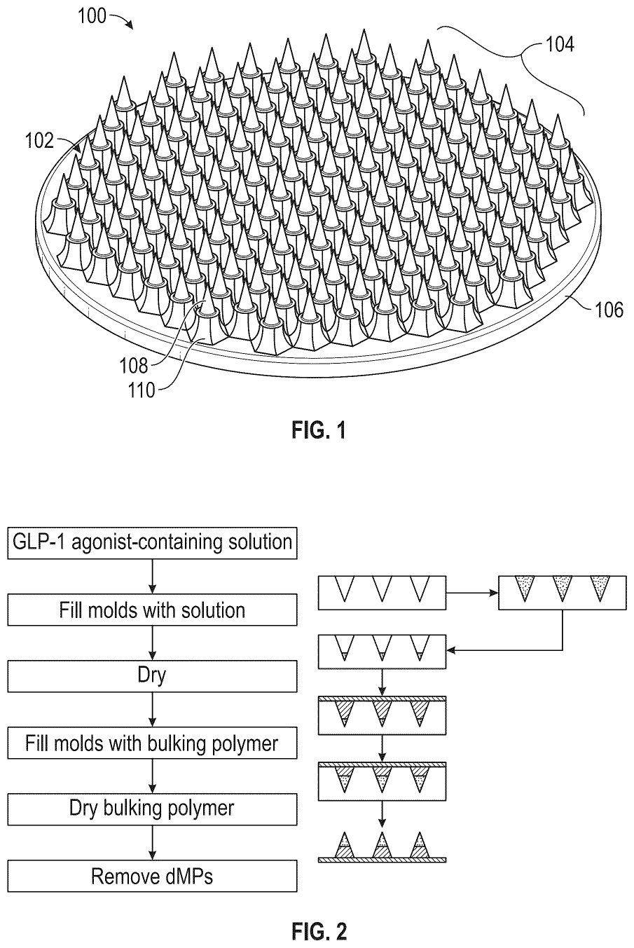

FIG. 1 illustrates one embodiment of a microneedle patch 100, which includes microneedles 102 provided in the form of a microneedle array 104 extending from a base 106. In this embodiment, each microneedle 102 has a conical tip end portion 108 that comprises a mixture of a GLP-1 agonist and an excipient composition as described herein, and a funnel portion 110 extending from and connecting the base 106 and the tip end portion 108. The funnel portion may be integrally formed with the base.

In some embodiments of using the microneedles described herein, a microneedle patch comprising the GLP-1 agonist-containing microneedles is applied to a tissue surface of a subject, such as a human subject or patient, in a manner to insert the microneedles, or at least the tip end portions thereof, into the subject's skin or other biological tissue. It is understood that following a period of being applied, the microneedle patch (e.g., its backing and any other parts remaining after the administration of the GLP-1 agonist and excipient composition) is removed from the tissue surface. The tissue may be mucosal or skin tissue. In one particular embodiment, the microneedle patch is applied to the skin, preferably on an arm or wrist, and is tolerated by the subject for the period of application of the patch. The period of application of the patch may be 5 minutes or less, preferably 1 minute or less. In some cases, during the period of the microneedle patch is applied to the tissue, the inserted microneedles, or at least the tip end portions thereof, may be substantially dissolved within the tissue. In preferred cases, the delivery efficiency of the GLP-1 agonist from the microneedle patch is at least 60%, more preferably at least 75%, for example from about 85% to about 95%.

The microneedles may be produced using any suitable method. In some embodiments, the microneedle is formed by (a) casting, or dispensing, into/onto a mold for the microneedle a liquid comprising a mixture of (i) a GLP-1 agonist; (ii) an excipient composition which comprises a carbohydrate and a polymer, and (iii) at least one solvent for excipient composition, and then (b) solidifying the cast liquid to remove at least enough of the solvent to form a solid microneedle composition which comprises the GLP-1 agonist dispersed in the excipient composition. An array of microneedles can be similarly prepared using a mold for an array of microneedles. The microneedle array may be part of a microneedle patch, which may further include a base layer cast onto a base side, i.e., proximal end portion, of the plurality of microneedles. After the cast/dispensed materials are solidified, the microneedles together with the base are removed from the mold.

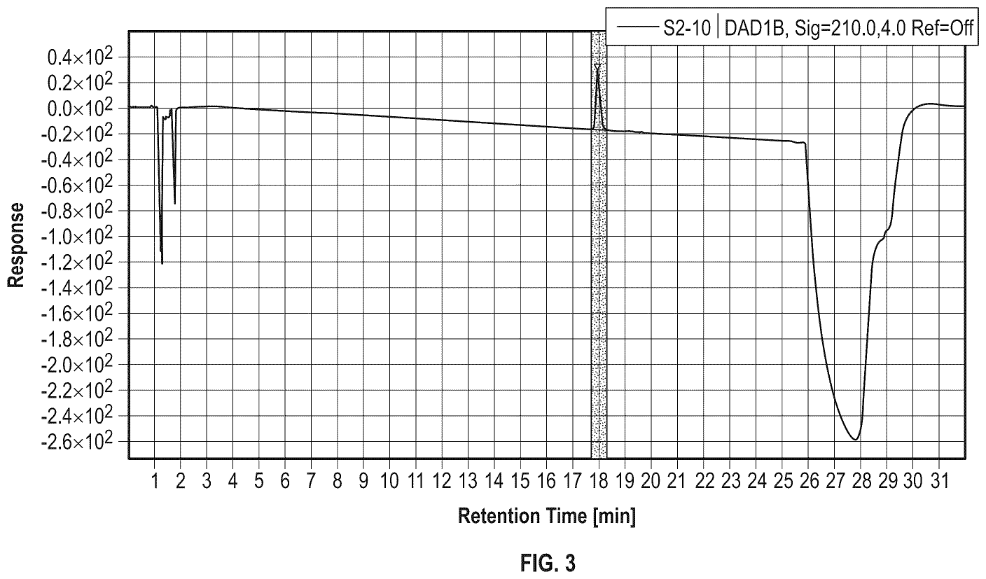

In some particular embodiments, the method includes a two-cast process for fabricating the microneedle, wherein the process includes: (a) preparing a first liquid which comprises (i) a GLP-1 agonist, (ii) an excipient composition containing a carbohydrate and a polymer, and (iii) a solvent; (b) casting the first liquid into or onto a mold; (c) solidifying the cast first liquid by removing at least enough of the solvent to form a tip end portion of the microneedle; (d) preparing a second liquid which is free of GLP-1 agonist; (e) casting the second liquid into or onto the mold containing the tip end portion of the microneedle; (f) solidifying the second liquid to form a proximal region of the microneedle which interfaces with the tip end portion. One embodiment of this process is illustrated in FIG. 2. In one embodiment, the second cast material may be a water-soluble formulation. In another embodiment, the second cast material is non-water-soluble after solidification. In some embodiments, the casting liquids are solidified via drying. In some other embodiments, the casting liquids are solidified via curing. In a specific embodiment, the second cast liquid is solidified via curing. The filing and solidifying steps may be referred to herein as “casting.” Examples of suitable methods are described in U.S. Pat. No. 10,828,478, which is incorporated herein by reference.

The microneedle formulations and microneedle arrays disclosed herein can be used to treat a subject. For example, the amount may be effective to treat or manage a metabolic condition, such as diabetes and/or obesity, or for other therapeutic uses, including aiding in weight loss and/or weight management, appetite suppression, and longevity.

The methods of using the microneedles may include applying to an area of tissue, for example, skin, of the subject a drug delivery device that includes an array of microneedles comprising a GLP-1 agonist formulation as disclosed herein, wherein the applying is effective to administer the GLP-1 agonist via the array of microneedles and to deliver a pharmaceutically acceptable amount of the GLP-1 agonist to the subject. The drug delivery device may be a microneedle patch applied to the skin, preferably of an arm or wrist, of the subject. The period of the application of the patch may be 5 minutes or less, preferably 1 minute or less.

In some embodiments, the method of administering the GLP-1 agonist may include identifying an application site on the skin and, preferably, sanitizing the area prior to application of the microneedle patch (e.g., using an alcohol wipe). If needed, the application site may be allowed to dry before application of the microneedle patch. The microneedle patch then is applied and pressed into the skin (e.g., using the thumb or finger) by applying a sufficient pressure/force to insert the microneedles. In other embodiments, the microneedle patches as disclosed herein can be applied by an external applicator to allow the microneedles to penetrate the biological tissue.

In some particular embodiments, the microneedle arrays contain at least 1 mg of a GLP-1 agonist and are thermostable for at least 1 month at a temperature of 5° C. to 40° C.

Advantageously, the methods with the current formulations may provide a delivery efficiency of the GLP-1 agonist that is at least 60%, 70%, 75%, 80%, 85%, 90% or 95%. For example, a microneedle array or patch with a delivery efficiency of approximately 80% may contain at least 1.25 mg of the GLP-1 agonist to achieve the delivery of at least 1 mg of the GLP-1 agonist to the subject.

In some embodiments, the method for administering a GLP-1 agonist to a subject may include applying to an area of tissue, for example, skin, of the subject a dissolvable microneedle patch (dMP) that contains at least 0.25 mg of the GLP-1 agonist. In some other embodiments, the dMP may contain at least 0.5 mg, 0.75 mg, 1 mg, 1.5 mg, 2 mg, or 2.5 mg of the GLP-1. The dMPs may be administered to the subject at any suitable dosage frequency to achieve the desired dose of the GLP-1 agonist over a period of time. In one embodiment, a dMP containing at least 0.25 mg, 0.5 mg, 1.0 mg, 1.5 mg, 2.0 mg, or 2.5 mg of a GLP-1 agonist is administered daily. In another embodiment, a dMP containing at least 0.25 mg, 0.5 mg, 1.0 mg, 1.5 mg, 2.0 mg, or 2.5 mg of a GLP-1 agonist is administered weekly. For particular therapeutic applications and/or for particular subjects, the dose amount of the GLP-1 agonist may be escalated over time, and/or de-escalate over time, for example as the subject adjusts to the medication or is being weaned off of the medication, e.g., in order to minimize undesirable side effects.

The microneedle patches described herein may be provided in kits with instructions. For example, a kit may contain two or more dMPs containing at least 0.01 mg of a GLP-1 agonist; and instructions for using the dMPs to treat a subject. The instructions may include the frequency of dosing and/or directions for administering the dMPs. In some embodiments, the GLP1-dMP is configured for self-administration by users in a simple and painless manner.

In some embodiments, after application of the microneedle patch for a period of time, the microneedles, or part thereof, containing a GLP-1 agonist may separate from their base. In some other embodiments, after application of the microneedle patch for a period of time, the microneedles containing a GLP-1 agonist may dissolve. For example, the microneedles are formed of a mixture including the GLP-1 agonist dispersed in a water-soluble matrix material (excipient composition), such that the microneedles are configured to dissolve in vivo following insertion. In some other embodiments, the microneedles may be configured to dissolve such that the GLP-1 agonist is delivered into the skin after a period of time. For example, the dissolution can be sufficient to separate the GLP-1-agonist containing distal tip end portion of the microneedle from the proximal region of the microneedle.

Unless otherwise defined herein, all technical and scientific terms used herein have meanings commonly understood by those of skill in the art to which the present invention pertains. It is also to be understood that the terminology used herein is for the purpose of describing particular embodiments only and is not intended to be limiting.

The term “about,” as used herein, indicates the value of a given quantity can include quantities ranging within 10% of the stated value, or optionally within 5% of the value, or in some embodiments within 1% of the value.

### Additional Details and Options

Features, options, and modifications of the microneedles, microneedle arrays, and methods described above are set forth with additional characterizations and details described below. Some details pertaining the microneedles arrays and their methods of manufacture may be described in U.S. Pat. Nos. 10,265,511; 10,828,478; 11,590,330; 10,940,301; 11,730,937, WO 2024/059152 and US20200238065A1, which are incorporated herein by reference.

Microneedle Patch Arrays The microneedle patch may include a base and an array of microneedles extending from the base, wherein at least a distal tip portion of each of the microneedles contains a GLP-1 agonist and excipient composition (comprising a carbohydrate and polymer mixture as described herein), wherein the distal tip portion of each microneedle is configured to be inserted into a subject, where the subject is a human, other mammal, or animal. The microneedles can each include a drug-free, or substantially drug-free, proximal portion between the base and the distal tip portion of the microneedles. The microneedle patches disclosed herein preferably are configured and formulated such that the distal tip portion of each microneedle will dissolve and/or separate from the base in less than 5 minutes, or, for example, between 1 minute and 5 minutes or less than 1 minute.

In certain embodiments, at least one primary funnel portion may be disposed between and connect the base and the microneedles. In another embodiment, a secondary funnel portion may extend from the primary funnel. In some embodiments, the microneedle arrays include a base having a microneedle side and an opposing back side; at least one primary funnel portion extending from the microneedle side of the base; and two or more solid microneedles extending from the at least one primary funnel portion, wherein the two or more solid microneedles include a tip end portion as described herein and a proximal funnel portion extends from the at least one primary funnel. The two or more solid microneedles may be constructed to penetrate into the subject's biological tissue under compression and then to separate from the secondary funnel portions or base substrate. In certain embodiments, the biological tissue can be skin or a mucosal membrane, such as an oral or nasal tissue.

The secondary funnel portion can facilitate insertion of the microneedles below the surface of the skin or other biological tissue, for example, so that essentially no part of the separated microneedle protrudes out of the biological tissue, which would for example, impede a proper and complete delivery of a dose of the substance of interest. However, in some other embodiments that result may be of little or no concern. Therefore, in some embodiments, the second funnel portions are omitted, and the microneedles extend directly from a primary funnel portion.

Each microneedle can be associated with one funnel and each funnel associated with one microneedle. Alternatively, one microneedle can be associated with more than one funnel. Alternatively, one funnel can be associated with more than one microneedle. In general, on a per patch basis, the number of microneedles is greater than or equal to the number of funnels. However, the number of funnels may exceed the number of microneedles when the funnels are used in series.

The number of microneedles per patch is generally between 1 and 10,000, and in most cases is between about 20 and 1000 and more preferably between about 50 and 500. In particular embodiments, there are 140, 150, 160, or 170 microneedles per patch. The number of funnels per patch is generally between about 1 and 10,000, and in most cases is between about 5 and 500 and preferably between about 10 and 500. The ratio of funnels to microneedles is between about 0.001 to 1, more typically between about 0.005 and 0.5 and more preferably between 0.001 and 0.008. In particular embodiments, the ratio is 0.006. In some cases, the ratio of funnels to microneedles is about 1. In other cases, the ratio of funnels to microneedles is about 2 or greater. In some cases, a plurality of microneedles all in a row is associated with the same funnel. In some cases, some of the microneedles are associated with funnels and other microneedles are not associated with funnels. In some cases, the number of funnels that each microneedle is associated with within a patch is not the same for all microneedles or for all funnels.

Funnels can also be used in series, i.e., a collection of funnels where the first funnel (i.e., a primary funnel portion) (base end) feeds a number of other funnels (i.e., secondary funnel portions). For example, each microneedle may have its own funnel and a row or section of a patch of microneedles and funnels may be connected to a larger elongated funnel.

The microneedle patches described herein may include components, such as adhesive layers, backing layers, handles/tabs, force feedback indicators, etc. that facilitate handling of the patches, manually with or without the aid of an applicator tool, without contacting the microneedle arrays, that protect the microneedles (or other components) from moisture, gases, and contaminants for example during storage or shipment, and/or that facilitate microneedle insertion into the subject.

In some embodiments, the microneedle patch may include microneedles that are made of different excipient compositions and/or include different amounts or types of GLP-1 agonists arranged within the same patch. In some embodiments, the microneedle patch may include a second pharmaceutical agent, which may be in the same or different microneedles as the GLP-1 agonist.

The microneedle arrays may contain at least 0.01, 0.05, 0.0625, 0.125, 0.25, 0.5, 0.75, 1.25, 1.5, 1.75, 2, 2.5, 3, 5, 7.5, 10, or 15 milligrams of a GLP-1 agonist. In particular embodiments, the microneedle arrays contain at least 1 milligram of a GLP-1 agonist. The microneedle arrays can be configured to allow for both the appropriate dose of a GLP-1 agonist to be administered and ease of application by a subject. In certain embodiments, the microneedle array has at least approximately 160 GLP-1 agonist-containing microneedles. In a specific embodiment, the microneedle array has 163 microneedles. In another embodiments, the microneedle array has at least 20, 25, 50, 100, 150, 200, 250, 300 or 350 microneedles. Each microneedle preferably is conical in shape and at least 300, 400, 500, 600, 700, 800, 900, 1000, or 1100 microns in length, although other shapes and dimensions are envisioned. The microneedles in the array can be arranged in a circular pattern and the diameter of the microneedle array can be at least 0.5, 1, 1.5, 2, 2.5, or 3 centimeters. In a particular embodiment, the microneedle array contains approximately 1.25 milligrams of a GLP-1 agonist, has about 160 microneedles that are about 700 microns in length and arranged in a circular pattern that has a diameter of about 1 centimeter. In a specific embodiment, the GLP-1 agonist is semaglutide.

The GLP-1 agonist-containing microneedles may be thermostable. In some embodiments, the thermostability is at least two weeks at 40° C. In some embodiments, the thermostability is at least four weeks, at least six weeks, at least eight weeks, at least twelve weeks, or at least twenty-four weeks at 40° C. In some other embodiments, the thermostability is at least 3, 6, 9, 12, 18, or 24 months at 5° C.

As used herein, the terms “matrix material” and “excipient” are used interchangeably when referring to any excipients that are not volatilized or otherwise removed during drying and formation of the microneedles and funnels.

GLP-1 agonists can be formulated with water-soluble excipients to form the microneedles as disclosed herein. The water-soluble excipients can include any known in the art. The excipients are preferably found in existing commercial drug products, such as those listed the FDA's Inactive Ingredients in Approved Drug Products database. The water-soluble excipient(s) can be a combination of sugar/sugar alcohol and a water-soluble polymer. The water-soluble matrix material can be, but is not limited to, dextran, natural polysaccharides, hyaluronic acid, chitosan, beta-sodium glycerophosphate, hydroxypropyl beta cyclodextrin and/or water-soluble polymers, such as poly (vinyl alcohol) (PVA), polyvinyl pyrrolidone (PVP) (Jiang et al. Acta Materia Medica 2023, Volume 2, Issue 1, p. 1-8). In a particular embodiment, the GLP-1 agonist can be encapsulated and/or formulated in microneedles made of highly water-soluble PVA wherein the PVA is dissolvable and not cross-linked.

The matrix material forms the bulk of the microneedle, funnel portion, and/or base layer. In embodiments, the matrix material can be water-soluble. In certain preferred embodiments, the matrix material includes one or a combination of polyvinyl alcohol, dextran, carboxymethylcellulose, maltodextrin, sucrose and other sugars.

In some embodiments, a selected material is resilient enough to allow for penetration of the biological tissue, such as skin or other mucosal membranes. In some embodiments, the dissolvable microneedles dissolve within seconds, such as within about 5, 10, 15, 20, 25, 30, 45, 50, 60, 120, 180, or more seconds. In some embodiments, the dissolvable microneedle dissolves within minutes, such as within about 1, 2, 3, 4, 5, 6, 7, 8, 9, 10, 15, 20, 30, 60, 120, or more minutes. In some embodiments, the dissolvable microneedle comprises a dissolvable portion (such as the distal tip of the microneedle) and a non-dissolvable portion (such as the more proximal base of a microneedle).

In certain embodiments, after application for a period of time, the dissolving microneedles containing a GLP-1 agonist will separate from their base.

In embodiments, the microneedles include a GLP-1 agonist and an excipient composition. In some embodiments, the excipient composition includes a sugar and a water-soluble polymer. In some embodiments, the GLP-1 agonist and the excipient composition are present in a 1:1, 1:2, 1:3, 1:6, 1:10 mass ratio. In some embodiments, the mass ratio of the sugar to the water-soluble polymer in the excipient composition is 1.5:1, 6:4, 7:3, or 8:2. For example, in certain embodiments, when the mass ratio of the GLP-1 agonist to excipient is 1:1, the mass ratio of sugar to PVA may be 7:3, 6:4, 8:2, or 1.5:1. In other embodiments, the mass ratio of the sugar to water-soluble polymer in the excipient composition is 1:1, 2:8 3:7, or 4:6. In other words, when the mass ratio of the GLP-1 agonist to excipient is 1:1, the GLP-1 agonist is present in the microneedle in an amount of 50% by weight. The sugar and water-soluble polymer may therefore be present in the microneedle in respective amounts of 35% and 15% by weight (7:3), 30% and 20% by weight (6:4), 40% and 10% by weight (8:2), or 25% and 25% by weight (1:1). In some embodiments, the excipient composition may include one or more additional ingredients in addition to the sugar and water-soluble polymer, where the one or more additional ingredients may be a buffer and/or glycerol. In a preferred embodiment, the water-soluble polymer is PVA. According to another preferred embodiment, the sugar is dextrose, the water-soluble polymer is PVA and the GLP-1 agonist is semaglutide (SG).

The excipient composition may be water soluble. In some embodiments, the excipient composition comprises polyvinyl alcohol (PVA) and maltose. In other embodiments, the excipient composition comprises polyvinylpyrrolidone (PVP), dextrose, and glycerol. In further embodiments, the excipient composition comprises polyvinyl alcohol (PVA), dextrose, and HEPES. In even further embodiments, the excipient composition comprises polyvinyl alcohol (PVA) and glucose. The excipient composition may comprise 30% by weight glucose and 70% by weight PVA, or 70% by weight glucose and 30% by weight PVA.

In some embodiments, the microneedle formulation includes 1 to 50% by weight GLP-1 agonist, 10-50% by weight water-soluble polymer (e.g., PVA or PVP), 25-70% by weight sugar or sugar alcohol, up to 10% by weight glycerol, and up to 30% by weight buffer salts (e.g., HEPES, CaCl, phosphate, etc.).

Additional sugars that can be used in the excipient composition include: fructose, galactitol, glucose, mannitol, mannose, sorbitol, xylitol, xylose, lactose, maltose, sucrose, trehalose, pullulan and/or chitosan. Other water-soluble excipients that can be used in the excipient composition include: dextran, maltodextrin, hyaluronic acid, caboxymethyl cellulose Na, methylcellulose, hydroxypropyl cellulose, hydroxypropyl methylcellulose, poly (2-ethyl-2-oxazoline), polyacrylic acid sodium salt, polyacrylic acid.

GLP-1 agonists as disclosed herein can be referred to by other names. It is envisioned that synonyms of GLP-1 agonists are considered within the scope of this invention. For example, GLP-1 agonists can be known as GLP, GLP agonists, Glucagon-like peptide-1 agonists, GLP-1 receptor agonists, Incretin mimetics and/or GLP-1 analogs.

In some embodiments, the GLP-1 agonist can be semaglutide, semaglutide analogues, dulaglutide, liraglutide, exenatide, tirzepatide, lixisenatide, albiglutide, or a combination thereof. GLP-1 agonist medications currently available on the U.S. market are also included according to the compositions and methods disclosed herein and include: Dulaglutide (Trulicity®), Exenatide (Byetta®), Exenatide extended-release (Bydureon®), Liraglutide (Victoza®), Lixisenatide (Adlyxin®), Semaglutide injection (Ozempic®), Semaglutide tablets (Rybelsus®), tirzepatide (Mounjaro®), tirzepatide (Zepbound).

The microneedle array, including the microneedles, funnels, and/or base may be made of the same or different materials. In certain embodiments, two casts can be used to form the dMP. The first cast can include a water-soluble excipient composition, such as a combination of sugar/sugar alcohol and a water-soluble polymer. The water-soluble excipient composition may include dextran, natural polysaccharides, hyaluronic acid, chitosan, beta-sodium glycerophosphate, hydroxypropyl beta cyclodextrin and/or water-soluble polymers, such as poly (vinyl alcohol) (PVA), polyvinyl pyrrolidone (PVP) (Jiang et al. Acta Materia Medica 2023, Volume 2, Issue 1, p. 1-8). In a particular embodiment, the microneedles can be made of highly water-soluble PVA. In additional embodiments, the first case can include one or a combination of polyvinyl alcohol, dextran, carboxymethylcellulose, maltodextrin, sucrose and other sugars. The second cast can also be formulated with such water-soluble materials or with non-water-soluble materials, such as a non-erodible or biodegradable polymer. In a specific embodiment, the second cast can also be formulated with the excipient composition of the first cast but without the GLP-1 agonist mixed therein. In one embodiment, the second cast can be a curable (for example, light, two-part with catalyst, heat, etc.) polymer or resin. In another embodiment, it can be a freeze-thaw material, for example, thermoplastic (for example, a low melting material. In some embodiments, the material can be polycaprolactone or a wax.

### Methods of Making the Microneedles

In additional embodiments, the method can include a two-cast process for fabricating a microneedle including: (a) preparing a casting liquid which comprises a mixture (e.g., a solution) of (i) a GLP-1 agonist, and (ii) an excipient composition containing a carbohydrate and a polymer, and (iii) a solvent; (b) casting the casting liquid into or onto a mold; (c) solidifying (e.g., drying or curing) the cast solution to remove at least enough of the solvent to form the distal region, for example the tip, of the microneedle; (d) preparing a second casting liquid which containing an excipient and a solvent; (e) casting the second casting liquid into or onto the mold containing the distal region of the microneedle; (c) solidifying the second cast solution to remove at least enough of the solvent to form the proximal region of the microneedle. In one embodiment, the second cast excipient can be a water-soluble formulation. In other embodiments, the second cast excipient can be a non-water soluble excipient. In some embodiments, the casting solutions can be solidified via drying. In other embodiments, the casting solution can be solidified via curing. In a specific embodiment, the second cast solution is solidified via curing.

The casting liquid for use in any of these methods may be prepared by combining selected amounts of the GLP-1 agonist, solvent, and one or more other pharmaceutically acceptable excipients. The “casting liquid” may be an aqueous solution with a GLP-1 agonist dispersed/suspended therein. In some embodiments, the solvent comprises water. In some embodiments, the solvent can include Tris. In some embodiments, the other excipients are selected to form a microneedle with the required mechanical properties (e.g., stiffness) to be effective to be insertable into skin, e.g., to penetrate the stratum corneum, or other tissues. In some embodiments, the additional pharmaceutically acceptable excipient included in the casting liquid includes a carbohydrate, such as a disaccharide or polysaccharide. For example, the carbohydrate may be methylcellulose, maltodextrin, lactose, trehalose, sucrose, and a combination thereof.

Any suitable means for drying the cast solution may be used. As used herein, the term “drying,” “dried,” or “dry” as it refers to the material in a mold refers to the material becoming at least partially solidified. The microneedles may be removed from the mold before being fully dried or after the microneedles are dried to an operational state. In some preferred embodiments, the drying is performed at a temperature from 4° C. to 40° C., preferably from 4° C. to 10° C. After drying, the solid formulation, or microneedle, is removed from the mold.

In a preferred embodiment, a two-step filling process is used to make microneedles, wherein the first filling step contains the casting liquid comprising the GLP-1 agonist, which substantially migrates into the microneedle tips during the drying process. This is followed by a second filling step and a subsequent drying or curing step. This second filling step contains the matrix material(s) that forms the base of the microneedles and funnels, if included, and may be overfilled to create the base layer or part of the base layer.

In a preferred embodiment, the first casting liquid includes the GLP-1 agonist and one or more suitable excipients, and the second casting liquid comprises one or more different components, not including the GLP-1 agonist. Instead, the second casting liquid may include one or more water-soluble or non-water soluble polymers, or a polymerizable (curable) precursor. The second casting liquid may include a suitable aqueous or organic solvent.

In a preferred embodiment, the GLP-1 agonist is loaded preferentially into the microneedles and their tips, and not into the proximal portions of the microneedles and funnel portions if provided.

The microneedle patches may be inspected and packaged to protect it from mechanical damage, moisture, light, oxygen, and/or microbial or other contamination before it can be used.

### Methods of Use

In one aspect of the present invention, methods of use are provided that can include applying the microneedle patch disclosed herein to a biological tissue, such as skin, to insert the array of microneedles, and releasing the GLP-1 agonist.

In some embodiments, the microneedle patch is applied to a tissue of the subject. (It is understood that following a period of being applied, the microneedle patch (e.g., it backing and other parts remaining after the administration of the GLP-1 agonist formulation) is removed from the tissue.) The tissue can be mucosal or skin tissue. In one particular embodiment, the microneedle patch is applied to the skin, preferably of an arm or wrist, and is tolerated by the subject for the period of application of the patch. The period of application of the patch may be 5 minutes or less, preferably 1 minute or less. The period of application of the patch may be 5 minutes or less, preferably 1 minute or less, or more preferably, 30 seconds or less or 10 seconds or less.

In additional embodiments, methods to treat a subject are provided in which the delivery efficiency of the GLP-1 agonist is at least 60%, 70%, 75%, 80%, 85%, 90% and/or 95%. For example, in a particular embodiment, a microneedle formulation with a delivery efficiency of approximately 80% contains at least 1.25 milligrams of a GLP-1 agonist to achieve the delivery of at least 1 milligram of the GLP-1 agonist to the subject.

In additional embodiments, a method is provided for treating a subject, wherein the method includes applying to an area of tissue, for example, skin, of the subject a dMP that contains at least 0.25 mg of a GLP-1 agonist in the microneedle compositions described herein. In other embodiments, at least 0.5, 0.75, 1, 1.5, 2, or 2.5 mg of a GLP-1 agonist is contained in a dMP as described herein. The dMPs may be applied/administered to the subject at frequencies to achieve a desired dose of the GLP-1 agonist over a period of time. In one embodiment, a dMP containing at least 0.25 mg, 0.5 mg, 1.0 mg, 1.5 mg, 2.0 mg or 2.5 mg of a GLP-1 agonist is administered daily. In another embodiment, a dMP containing at least 0.25 mg, 0.5 mg, 1.0 mg, 1.5 mg, 2.0 mg or 2.5 mg of a GLP-1 agonist is administered weekly. The dose of the GLP-1 agonist-containing dMPs may be escalated over time or de-escalated over time.

It is envisioned that the methods and kits to administer the DLP1-dMPs provided herein can contain a single dose of a GLP-1 agonist, for example, between 0.1 and 1.5 mg, wherein one or more of the patches can be applied per week. The patches can be applied at least once or more per week and the dose can be escalated. In addition, the method or instructions in the kit, may provide, for example, that one patch per week treats or manages one disease or condition and a different number of patches/week, for example, 2, 3, or 4 or more, treats or manages a different disease or condition. In a specific example, the method or instructions in the kit, may provide, for example, that one patch per week treats diabetes and up to 3 patches/week treat suppress appetite/weight loss for a particular dose or doses of a GLP-1 agonist. For example, the diabetes dose may be at least 1 mg weekly whereas the weight loss dose may be at least 2.4 mg weekly.

In non-limiting embodiments, the methods and kits provided herein for the GLP-1 dMPs can be according to the dosing schedule of currently approved GLP-1 agonists or GLP-1 agonists in clinical development. Examples are set forth in Table A below.

| | | Dosing | |
|---|---|---|---|
| Drug | Brand Name(s) | Frequency | Dose Range |
| Semaglutide | Ozempic ® | Weekly | 0.25 mg* → 0.5 mg → 1 |
| | (T2DM), | | mg → 2 mg (Ozempic); 1.7 |
| | Wegovy ® | | mg → 2.4 mg (Wegovy) |
| | (Obesity) | | |
| Liraglutide | Victoza ® | Daily | 0.6 mg* → 1.2 mg → 1.8 |
| | (T2DM), | | mg (Victoza); up to 3 mg |
| | Saxenda ® | | (Saxenda) |
| | (Obesity) | | |
| Dulaglutide | Trulicity ® | Weekly | 0.75 mg* → 1.5 mg → 3 |
| | | | mg → 4.5 mg |
| Exenatide | Byetta ® (BID), | Twice | 5 mcg → 10 mcg (Byetta); |
| | Bydureon ® | daily or | 2 mg/week (Bydureon) |
| | (Weekly) | weekly | |
| Tirzepatide | Mounjaro ® | Weekly | 2.5 mg* → 5 mg → 7.5 |
| (dual GIP/ | (T2DM), | | mg → 10 mg → 12.5 |
| GLP-1) | Zepbound ® | | mg → 15 mg |
| | (Obesity) | | |

Oral GLP-1 Agonist

| | | Dosing | |
|---|---|---|---|
| Drug | Brand Name | Frequency | Dose Range |
| Semaglutide (oral) | Rybelsus ® | Daily | 3 mg* → 7 mg → 14 mg |

*Asterisked doses are starting doses.

The microneedle arrays may contain at least 0.01, 0.05, 0.0625, 0.125, 0.25, 0.5, 1.0, 1.5, 2.0 or 5 milligrams of a GLP-1 agonist. In particular embodiments, the microneedle arrays contain at least 1 milligram of a GLP-1 agonist. The microneedle arrays may be configured to allow for both the appropriate dose of a GLP-1 agonist to be administered and ease of application by a subject. In certain embodiments, the microneedle arrays can contain at least 25, 50, 100, 150, 300, 400 and/or 500 GLP-1 agonist-containing microneedles. Each microneedle may be conical in shape and at least 300, 400, 500, 600, 700, 800 or 900 microns in length. The microneedles in the array can be arranged in a circular or substantially circular pattern and the diameter of the microneedle array can be at least 0.5, 1, 1.5 or 2 centimeters. In a particular embodiment, the microneedle array can contain approximately 1.25 milligrams of a GLP-1 agonist, approximately 160 microneedles that are approximately 700 microns in length and arranged in a circular pattern that has a diameter of approximately 1 centimeter. In preferred embodiments, the microneedle arrays can contain 0.25-2.5 mg/cm² of a GLP-1 agonist. In particular embodiments, the microneedle arrays can contain 0.5-2 mg/cm² of a GLP-1 agonist. In other particular embodiments, the microneedle arrays can contain 0.5-1 mg/cm² of a GLP-1 agonist. For specific illustrative examples of GLP-1 dose, microneedle array diameter, microneedle array area and GLP-1 dose, see Table B below. The kits and methods provided herein allow for the efficient and effective dosing of various amounts of GLP-1 agonists.

TABLE B
Bolded and italics text indicate particular exemplary
dose (mg) of GLP-1 per array area (cm²)

| GLP-1 | Microneedle | Microneedle | GLP-1 |
|---|---|---|---|
| Dose | Array | Array | Dose |
| (mg) | Diameter (cm) | Area (cm²) | (mg/cm²) |
| 2.4 | 1.5 | 1.767 | 1.36 |
| 1.2 | 1.5 | 1.767 | 0.68 |
| 0.25 | 1.5 | 1.767 | 0.14 |
| 0.1 | 1.5 | 1.767 | 0.06 |
| 0.05 | 1.5 | 1.767 | 0.03 |
| 0.01 | 1.5 | 1.767 | 0.01 |
| 1.2 | 1 | 0.785 | 1.53 |
| 0.25 | 1 | 0.785 | 0.32 |
| 0.1 | 1 | 0.785 | 0.13 |
| 0.05 | 1 | 0.785 | 0.06 |
| 0.01 | 1 | 0.785 | 0.01 |
| 0.25 | 0.5 | 0.196 | 1.28 |
| 0.1 | 0.5 | 0.196 | 0.51 |
| 0.05 | 0.5 | 0.196 | 0.26 |
| 0.01 | 0.5 | 0.196 | 0.05 |

### Microdosing/Fractional Dosing

In another aspect of the microneedles, microneedle arrays, and microneedle patches described herein, methods and kits are provided to treat a subject by microdosing or fractional dosing of a GLP-1 agonist. Microdosing refers to the practice of using doses of GLP-1 agonists that are lower, sometimes significantly lower, than doses that are standard or recommended by clinicians or the drug manufacturer. The methods provided herein to treat a subject by microdosing by administering the GLP1-dMPs of the present invention can: reduce side effects, for example, nausea, vomiting or other gastrointestinal discomfort and reduce the cost associated with treatment. Methods provided herein can also be used to manage mild weight loss, manage cravings, metabolic health or cognitive support in a subject. These methods can increase or escalate the dose of the GLP-1 agonist gradually over time to allow the body to adjust and minimize side effects. In addition, the dose of the GLP-1 agonist can be de-escalated over time, i.e., the doses of GLP-1 agonist delivered using the disclosed microneedles and patches can be decreased over time. Microdosing kits and methods may include dMPs containing approximately 0.01, 0.0125, 0.05, 0.0625, 0.075, 0.125, 0.25, 0.5, 1.0, 1.5, 2.0, or 5 milligrams of a GLP-1 agonist.

The microneedle arrays and patches provided herein may be self-administered or administered by another individual (e.g., a parent, guardian, minimally trained healthcare worker, expertly trained healthcare worker, and/or others). In some embodiments, the microneedle patches described herein are used to deliver the therapeutic agent into the skin by inserting the microneedles across the stratum corneum (outer 10 to 20 microns of skin that is the barrier to transdermal transport) and into the viable epidermis and dermis. The small size of the microneedles enables them to cause little to no pain and target the intradermal space. The intradermal space is highly vascularized and rich in immune cells and provides an attractive path to administer therapeutics.

In a preferred embodiment, the microneedles are dissolvable and once in the intradermal space they dissolve within the interstitial fluid and release the therapeutic agent into the skin. Once the skin-penetrating portion (i.e., the tip end portion) of the microneedles are essentially fully dissolved, which may take less than 5 minutes, the patch (base, etc.) can be removed and discarded as non-sharps waste since the microneedles have dissolved away.

The microneedle patches may be used in fractional dosing methods. For example, a patient may use the dMPs to administer lower doses of the GLP-1 agonist than prescribed (e.g., to minimize side effects, tame food cravings, or any other reason) and administering these lower doses as they deem appropriate for themselves. Accordingly, in some embodiments, kits are provided that include dMPs with different doses, or fractional doses, which enable the patient to select, for example, a larger dose for diabetes and a lower dose at a later point for appetite suppression. A kit could include a plurality of fractional dose dMPs which are the same, and the patient could administer two or more patches to get a desired full dose. Kits could include combinations of full and fractional dose dMPs.

### EXAMPLES

The present invention may be further understood with reference to the following non-limiting examples.

### Example 1

### Quantification of Semaglutide Content

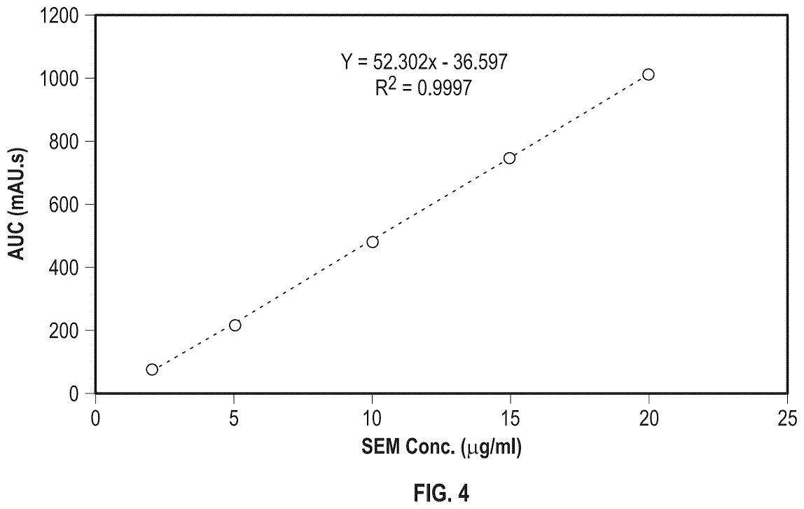

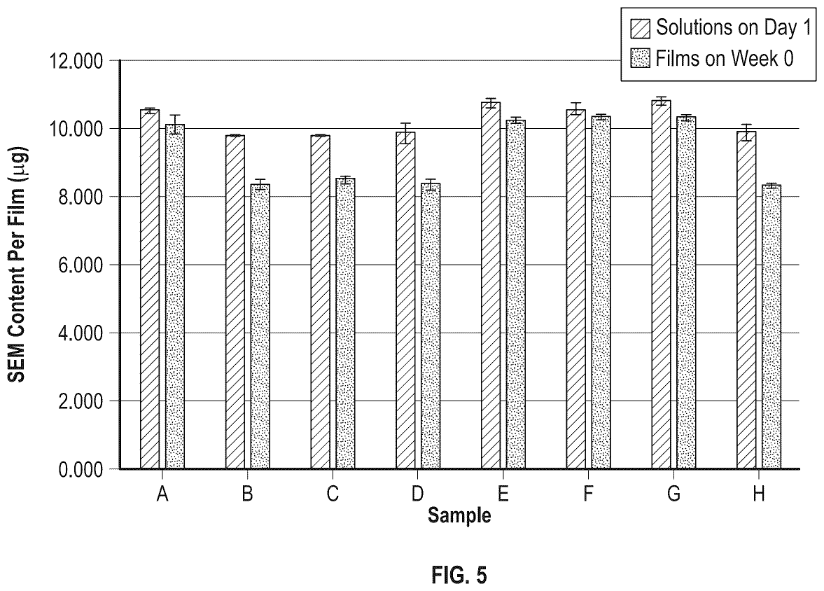

Liquid chromatography was used to detect a semaglutide analog (SG) in sample formulations, where a mixed solution of water and an organic solvent was used as a mobile phase A, an organic solvent was used as a mobile phase B, and a Charged Surface Hybrid (CHS) C18 reverse phase chromatography column was used as a stationary phase and an ultraviolet detector. Gradient elution of SG was carried out with these components, and it was observed that the main semaglutide peak eluted at around 18 minutes (FIG. 3). SG was dissolved in deionized (DI) water to prepare a 1 mg/mL stock solution. The stock solution was used to prepare calibrators in water. A calibration curve with a working range of 2-20 micrograms (mcg)/mL of SG was established using area under the curve (AUC) versus SG concentration (FIG. 4). This line fit of the calibration curve was then used to quantify the SG content of a liquid sample.

### Example 2

### Film Formulation Screening

The effect of polyvinyl alcohol (PVA), sugars, and sugar alcohols on the stability of SG during drying was evaluated. SG formulations having a 40:1 excipient to active ratio were prepared. Single excipient formulations contained 0.05% SG and 2% of the excipient. Dual excipient formulations contained 0.05% SG and 1% of each excipient. The complete listing of tested formulations is set forth in Table 1.

TABLE 1
Formulation screen solution composition.

| Code | Formulation |
|---|---|
| A | 0.05% SG/2% PVA |
| B | 0.05% SG/2% Sucrose |
| C | 0.05% SG/2% Sorbitol |
| D | 0.05% SG/2% Glucose |
| E | 0.05% SG/1% PVA/1% Sucrose |
| F | 0.05% SG/1% PVA/1% Sorbitol |
| G | 0.05% SG/1% PVA/1% Glucose |
| H | 0.05% SG only |

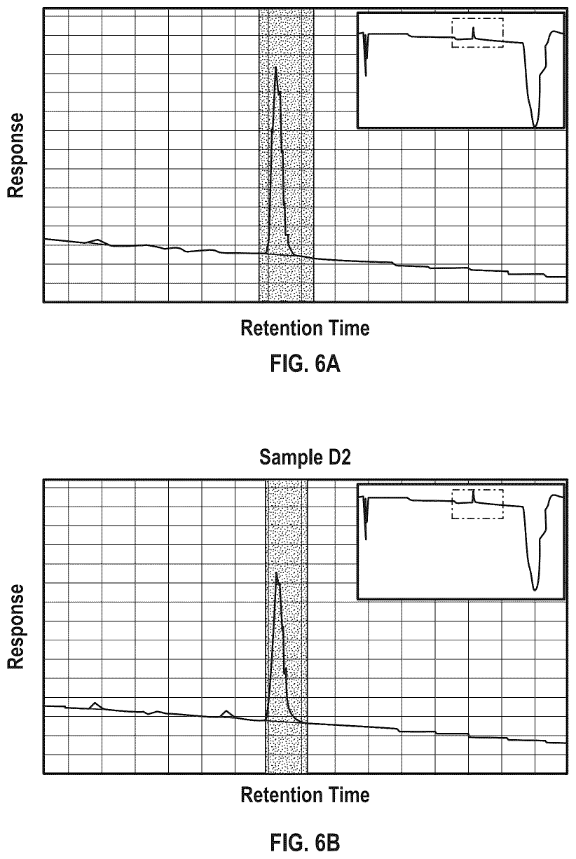

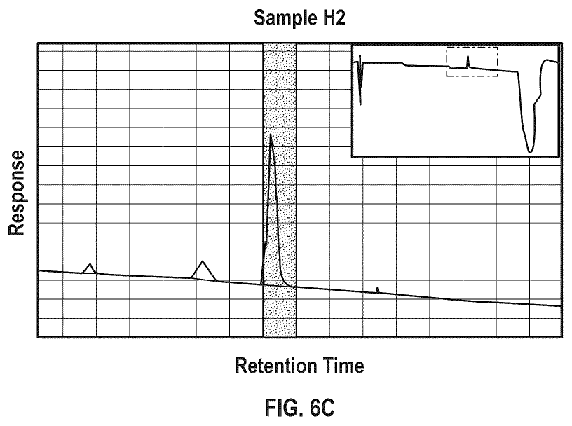

SG formulations in film form were prepared by depositing 20 mcL of solution on a polydimethylsiloxane (PDMS) substrate, and drying the solution under ambient conditions with airflow (e.g., in a biosafety cabinet) overnight. The liquid samples used to prepare the film formulations were refrigerated. The SG content of the liquid samples used to prepare the films was compared to the SG content present on the films after reconstitution. The PVA containing film formulations showed the highest recovery of SG (FIGS. 5-6C).

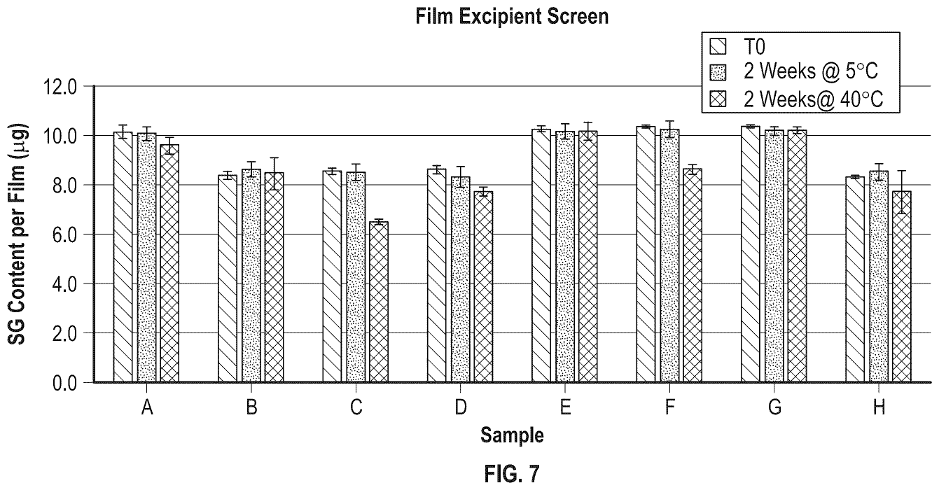

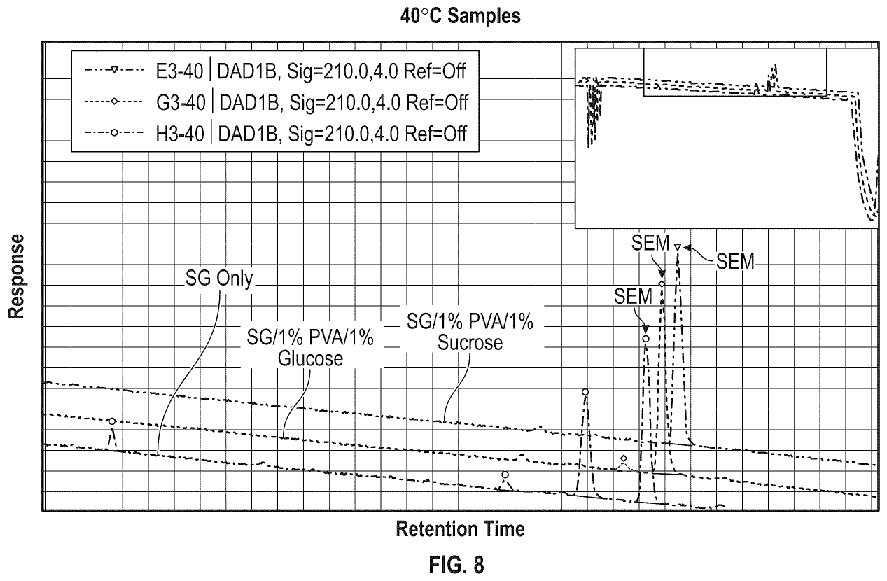

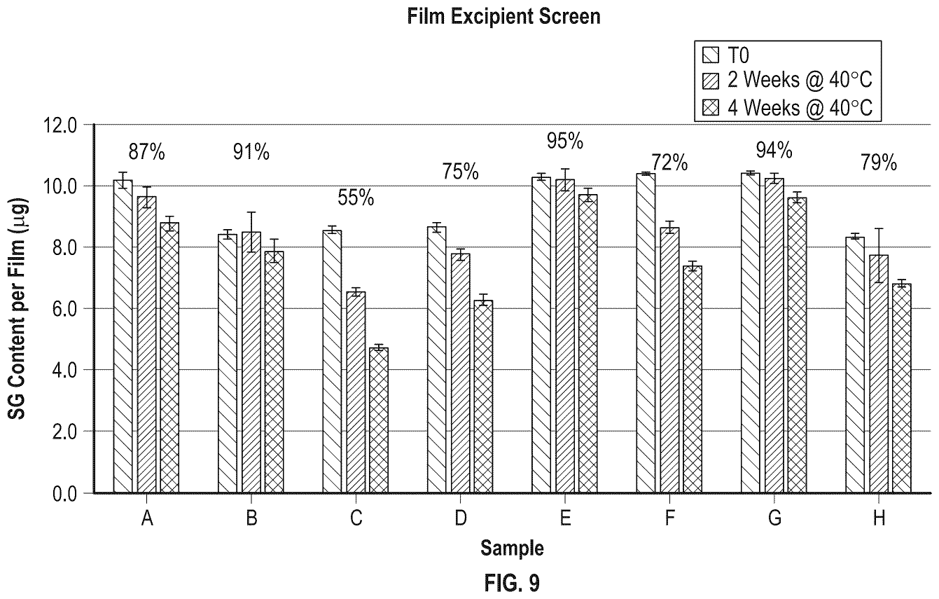

To evaluate the effect of the selected excipients on semaglutide stability over time, film samples were stored at 2-8° C. and 40° C. for up to 4 weeks. After 2 weeks of storage under refrigerated conditions, the formulations showed little to no loss in SG content compared to the SG content observed at T0 (FIG. 7). After 2 weeks of storage at 40° C., single excipient formulations with sucrose and PVA retained 98% and 95% of SG content compared to T0, respectively. Dual excipient formulations containing PVA and sucrose and PVA and glucose out-performed all single excipient formulations, showing no decrease in SG content over time. The improved stability of these formulations was also evident in the absence of degradation peaks in these formulations (FIG. 8). The PVA and sucrose and PVA and glucose formulations retained 95% and 94% of activity after 4 weeks of storage at 40° C., respectively (FIG. 9).

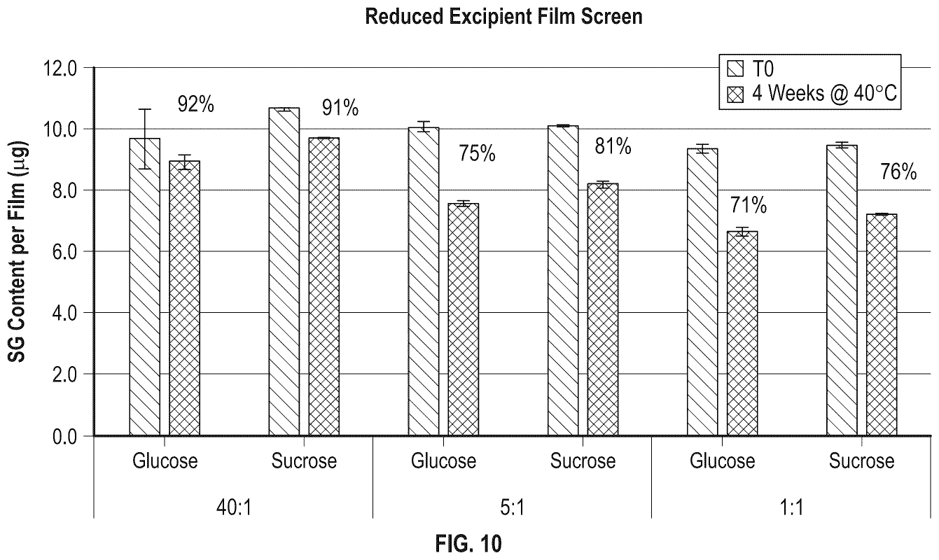

To increase SG content and decrease the total mass of the microneedle arrays, lower excipient to active ratio formulations were evaluated for stability, where the formulations included excipient compositions having a mixture of (i) PVA and sucrose and (ii) PVA and glucose. Films containing 10 mcg of SG were prepared by depositing and drying 20 mcL of solutions containing 0.05% SG and either 1% (40:1), 0.125% (5:1) or 0.025% (1:1) w/v of each excipient composition in water (FIG. 10).

### Example 3

### Dissolvable Semaglutide Microneedle Patch Development

The (i) PVA and sucrose and (ii) PVA and glucose excipient compositions were selected for the development of SG dissolving microneedle patches (SG-dMPs). Dissolving microneedle patches (dMPs) were fabricated from polydimethylsiloxane (PDMS) molds using a two-step casting method, as shown in FIG. 2. The process consisted of depositing and drying a SG-containing solution into a PDMS mold, followed by depositing and drying a polymer matrix solution within the mold on top of the dried SG solution. The resulting microneedle arrays contained 163 microneedles that were conical in shape and 700 μm tall.

For the initial evaluation, a 40:1 excipient to active ratio was used. 40 mcL of a 0.05% SG/1% PVA/1% sugar formulation was deposited into a PDMS mold and dried. The SG formulation also contained Sulforhodamine B to facilitate visualization of the deposition and drying of the formulation. After the first cast of the SG formulation was dried, 125 mcL of 18 wt % PVA/18 wt % sucrose or 18 wt % PVA/18 wt % glucose bulking polymer solution was deposited into the mold to form the vase of the microneedle array. After the bulking polymer solution was dried, the SG-dMPs were removed from the mold and evaluated for dose, microneedle functionality, and delivery efficiency.

### Semaglutide Content and Dose

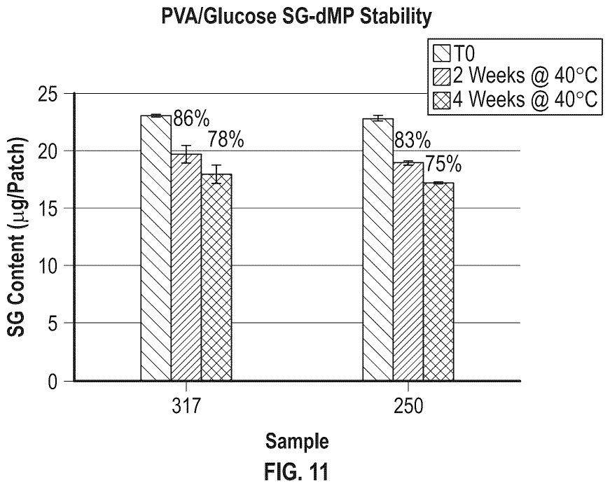

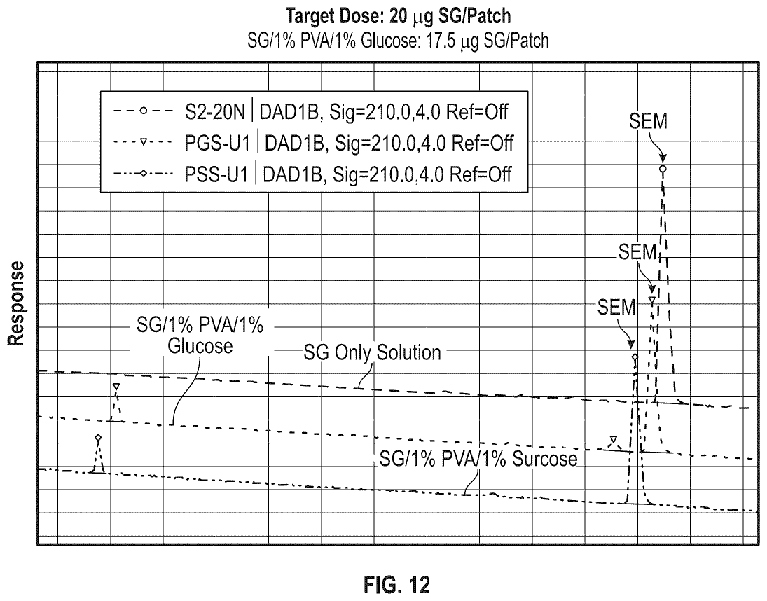

When analyzed for SG content, the PVA and glucose patches with a target dose of 20 mcg per patch contained an average of 17.5 mcg of SG (FIG. 11). New, minor peaks were observed in the chromatograms of the SG-dMPs, which were not present in the SG solution (FIG. 12).

### SG-dMP Functionality

To evaluate microneedle functionality, namely the ability of the microneedles to penetrate the skin and readily dissolve, microneedle arrays were inserted into excised defatted and dehaired porcine skin, and subsequently inspected under the microscope to evaluate microneedle dissolution. The skin was stained with gentian violet to visualize the microneedle penetration points after the SG-dMP was removed from skin. The previously described SG-dMP manufacturing process resulted in robust microneedles that penetrated the porcine skin and readily dissolved.

### Semaglutide Delivery Efficiency

In addition to dose and microneedle functionality, delivery efficiency (i.e., the percentage of the semaglutide dose delivered from the SG-dMP) and stability of the SG-dMPs were evaluated. SG-dMPs having PVA and glucose excipient compositions with an excipient to active ratio of ˜10:1 were used to manufacture SG-dMPs with a target dose of 25 mcg. Keeping semaglutide and glucose content constant, two different PVA concentrations were evaluated. Both formulations behaved similarly (FIG. 11).

TABLE 2
SG-dMP (dissolving base) content and stability.

| | | Dose | | | |
|---|---|---|---|---|---|
| Array Total | Dose | Delivered | Delivery | Stability at 40° C. | |
| Mass | (n = 2) | (n = 2) | Efficiency | 2 Weeks | 4 Weeks |
| 250 mcg | 22.9 mcg | 17.0 mcg | 74% | 18.9 mcg | 17.9 mcg |
| | | | | (83%) | (78%) |
| 317 mcg | 23.1 mcg | 17.5 mcg | 76% | 19.7 mcg | 17.3 mcg |
| | | | | (86%) | (75%) |

### Stability of SG-dMPs

Higher dose SG-dMPs with a 5:1 excipient to active ratio were also evaluated for stability. These patches had a target dose of 500 mcg of SG and were made by depositing and drying 40 mcL of a 1.25% SG/3.125% PVA/3.125% glucose solution, followed by the depositing and drying a second cast of a 18 wt % PVA/18 wt % glucose polymer in water solution to form a dissolving base for the dMPs.

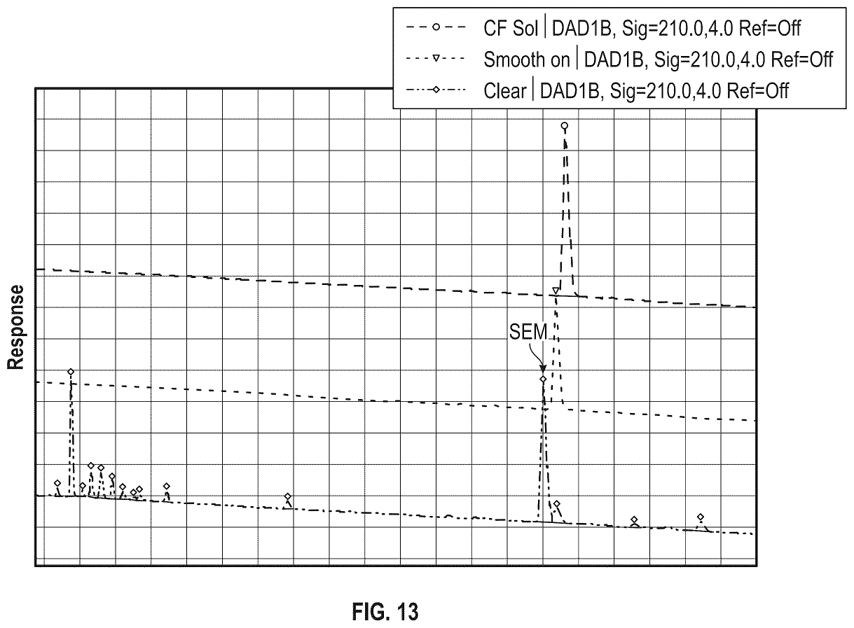

The SG-dMP manufactured with a target dose of 20 mcg of SG and an excipient composition having PVA and glucose was also evaluated using both a two-part curable urethane material and a photocurable medical grade resin to form the base of the dMPs. Both materials, once cured, are not soluble in water and do not dissolve during patch application. Although the two-part curable urethane displayed a lower dose and the presence of additional peaks on the chromatogram, the photocurable medical grade resin performed similarly to the arrays formed with a dissolving polymer base material (FIG. 13).

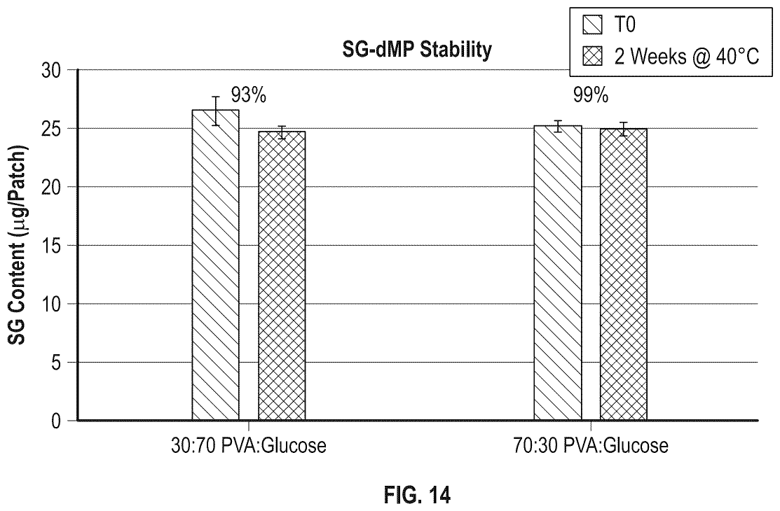

In another experiment, dMPs with a polycaprolactone (PCL) base, deposited as a melt that solidifies as it cools to room temperature, was used to form the base of the dMPs, and dMPs having higher excipient concentrations were also manufactured and evaluated. SG-dMPs with a 20:1 active ratio of excipient to SG and a target dose of 25 mcg were assessed for SG content over time. Keeping the total mass constant at 525 mcg, two ratios of PVA and glucose were evaluated. A high sugar formulation contained glucose and PVA at a 70:30 ratio and a high polymer formulation contained glucose and PVA at a 30:70 ratio were each assessed (FIG. 14).

TABLE 3
SG-dMP (PCL base) content and stability.

| | Array Total | Dose | Stability 2 Weeks |
|---|---|---|---|
| PVA:Glucose | Mass | (n = 2) | at 40° C. |
| 30:70 | 525 mcg | 26.4 mcg | 24.6 mcg (93%) |
| 70:30 | 525 mcg | 25.1 mcg | 24.8 mcg (99%) |

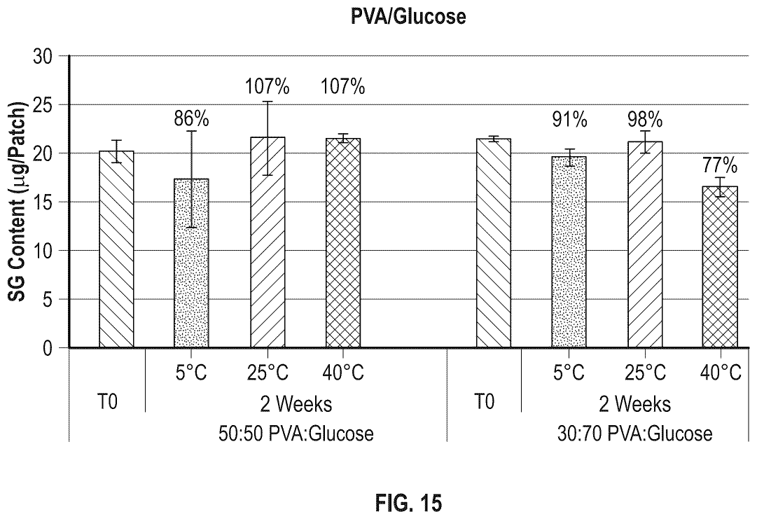

The stability of SG-dMPs formulated with a PVA and glucose excipient composition with a higher excipient to active ratio was also evaluated. Arrays containing 25 mcg of SG and 1 mg of excipients (40:1 excipient to active ratio) were prepared using PCL as the dMP base. The SG-dMPs were stored at 5° C., 25° C., and 40° C. The excipient compositions contained a 50:50 PVA:glucose ratio and 30:70 PVA:glucose ratio (low polymer content). Both formulations retained stability after 2 weeks of storage at 5° C. and 25° C. (FIG. 15).

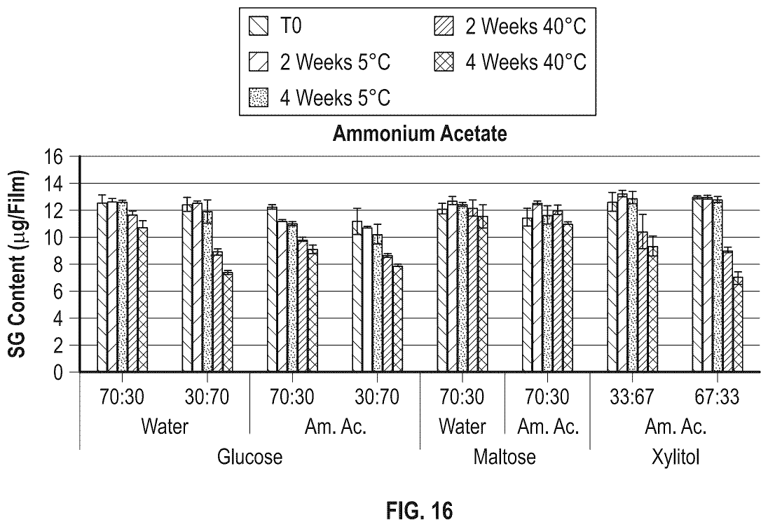

The effect of buffers and pH on SG stability in a SG-dMP was studied. In addition to the lead formulation containing glucose, formulations containing maltose and xylitol were also studied. Formulations containing either a high polymer with a 70:30 PVA:sugar ratio or a high sugar content with a 70:30 PVA:sugar ratio were prepared with a 30:1 excipient to active ratio, containing 12.5 mcg of SG and 375 mcg of excipients. Xylitol was evaluated at a 48:1 excipient to active ratio, containing 12.5 mcg of SG and 600 mcg of excipients, with the high polymer formulation containing a 67:33 PVA:xylitol ratio and the low polymer formulation containing a 33:67 PVA:xylitol ratio. Ammonium acetate did not seem to significantly improve SG stability, but the maltose formulations showed improved stability compared to glucose and xylitol (FIG. 16).

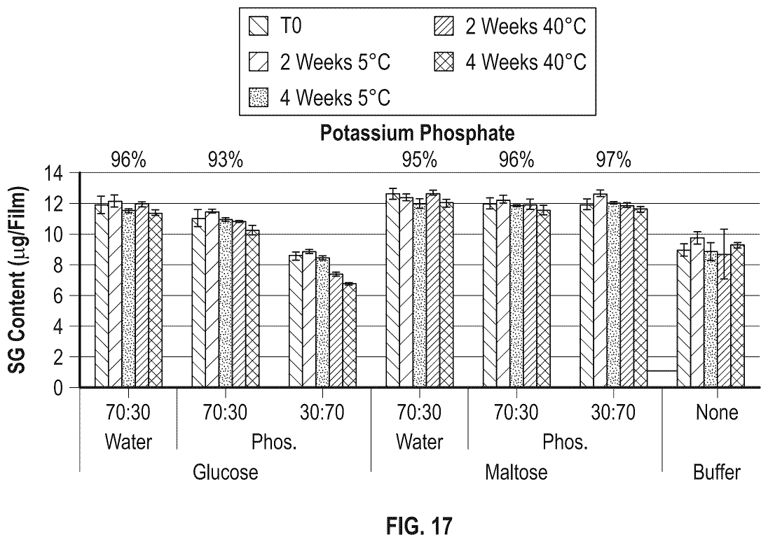

To evaluate the effect of potassium phosphate buffer on the stability of SG, formulations containing either a high polymer content with a 70:30 PVA:sugar ratio or high sugar content with a 30:70 PVA:sugar ratio were prepared with a 40:1 excipient to active ratio, containing 12.5 mcg of SG and 500 mcg of excipients. Formulations containing buffer only retained stability after 4 weeks of storage at 40° C. but showed significant loss upon drying (T0). (FIG. 17)

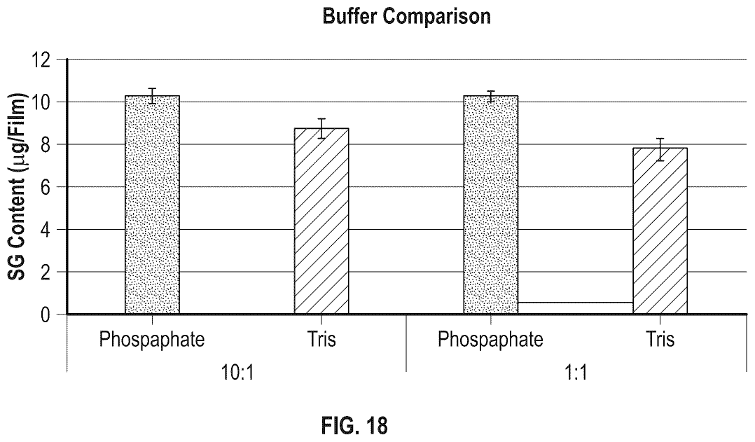

Tris-HCl buffer was also evaluated as a potential stabilizer. Films containing 10 mcg of SG and a 10:1 excipient to active ratio with an excipient composition of 30:70 PVA:glucose were prepared in 10 mM Tris-HCl buffer (pH 7) and compared to the same formulation prepared in 0.1 M phosphate buffer (pH 7). These formulations contained a 1:1 excipient to active ratio. The phosphate buffer outperformed Tris-HCl regardless of excipient to active ratio (FIG. 18). It also exhibited less loss upon drying. At a 1:1 ratio, the phosphate formulation retained ˜30% more SG than the Tris-HCl formulation.

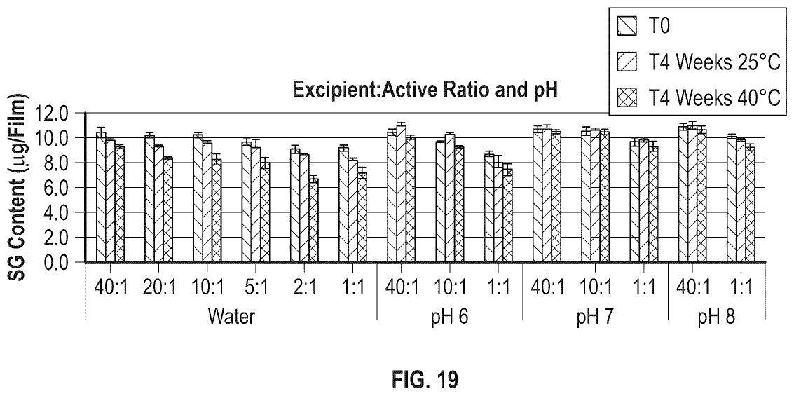

The effect of different excipient to active ratios in water was also evaluated. Films containing 10 mcg of SG and 400 mcg, 200 mcg, 100 mcg, 50 mcg, 20 mcg and 10 mcg of excipient compositions with 70:30 PVA: maltose were prepared and stored at 25° C. and 40° C. for up to 4 weeks. Additionally, several of these ratios were evaluated in formulations prepared in 0.1 M phosphate buffer across the pH range of 6 to 8. Formulations containing higher excipient concentrations exhibited lower loss of SG content during drying and over time, and formulations prepared in phosphate buffer (pH 7) outperformed the other formulations (FIG. 19). Films containing a 1:1 excipient to active ratio retained 96% of the SG content from T0 after 4 weeks of storage at 40° C., while formulations in water, buffer at pH 6, and buffer at pH 8 retained 78%, 86%, and 91% of the SG content, respectively.

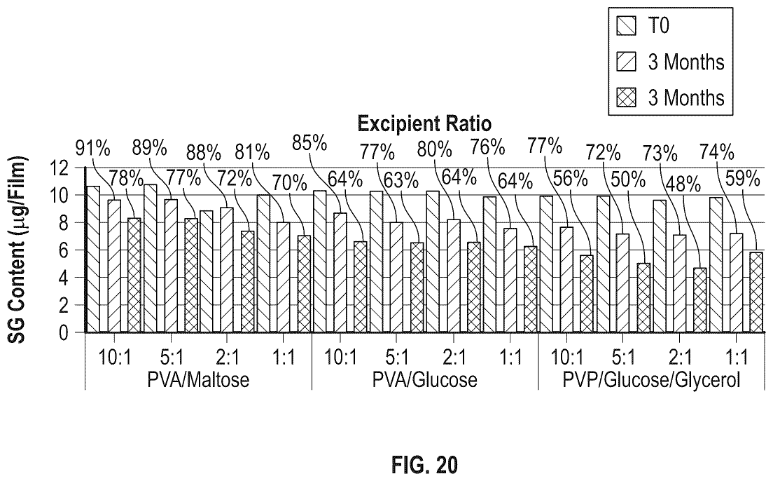

In addition to the 30:70 PVA: maltose excipient composition, formulations containing 30:70 PVA: glucose and 54:36:9:1 polyvinylpyrrolidone (PVP): glucose glycerol: tween in 0.1M phosphate buffer (pH 7) were also evaluated for stability at 10:1, 5:1, 2:1 and 1:1 excipient to active ratios (FIG. 20).

### Example 4

### Pharmacokinetic (PK) Study of Semaglutide Dissolvable Microneedle Patches

SG-dMPs were prepared to support a PK study in rats. An excipient composition containing a 20:1 excipient to active ratio of 20:80 PVA: maltose was used for the study. To deliver 20 mcg of SG, patches were manufactured with a target dose of 28 mcg, to account for a 10% loss on drying and an estimated delivery efficiency of 80%. Patches were made by depositing and drying 30 mcL of SG formulation solution, followed by depositing and drying 125 mcL of 18 wt % PVA/18 wt % maltose polymer solution in water. The average patch dose was calculated at 28.3±0.6 mcg of SG and the average dose delivered was 24.3±0.8 mcg, with an average delivery efficiency of 86%.

TABLE 4
PK study dose delivered and delivery efficiency.

| Animal | Dose Delivered (mcg) | Delivery Efficiency (%) |
|---|---|---|
| Rat 1 | 23.2 | 82% |
| Rat 2 | 25.2 | 89% |
| Rat 3 | 23.9 | 84% |
| Rat 4 | 24.7 | 87% |
| Rat 5 | 24.6 | 87% |

| Average | % RSD |
|---|---|
| 24.3 | 3.2 |

### Example 5

### Stability and Thermal Sterilization of Semaglutide Dissolvable Microneedle Patches

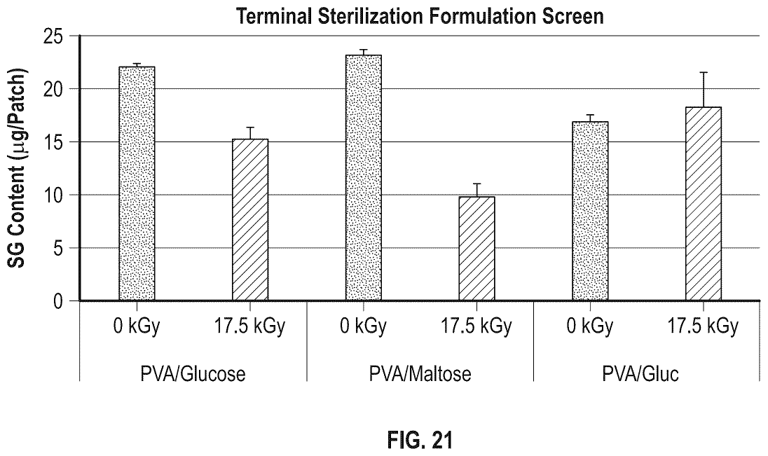

SG-dMPs with lead formulations were manufactured to evaluate formulation compatibility with gamma irradiation. Patches containing 25 mcg of SG and 750 mcg of excipients (30:1 excipient to active ratio) with a 30:70 PVA:sugar excipient composition were made by depositing and drying 40 mcL of the SG formulation into PDMS molds. The PVA and sugar excipient composition was used to make dMPs with a dissolving polymer matrix base consisting of 18% PVA/18% sucrose, and polycaprolactone (PCL) was used to make dMPs with a non-dissolving base. The PVA and maltose excipient composition was only evaluated with a dissolving base consisting of 30 wt % maltose/6 wt % PVA. The SG-dMPs were treated with 17.5-28 kGy of gamma irradiation and subsequently tested for SG content. The irradiation treatment did not cause any visible changes to the SG content of the SG-dMPs formulated with glucose (FIG. 21).

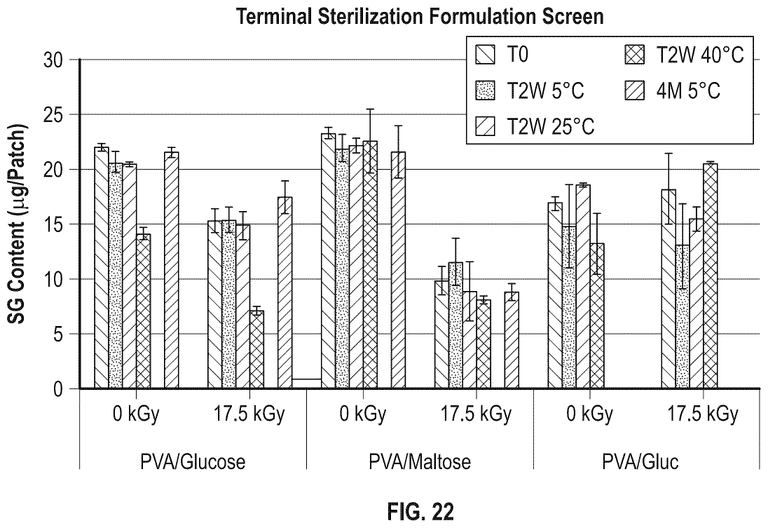

The irradiated SG-dMPs were then stored under various conditions and assessed for stability over time (FIG. 22). The SG-dMPs with both dissolving base formulations retained potency after 2 weeks of storage at 5° C. and 25° C. The highest decrease in content was observed after treatment, not during storage. The patches remained stable for up to 4 months under refrigerated storage conditions.

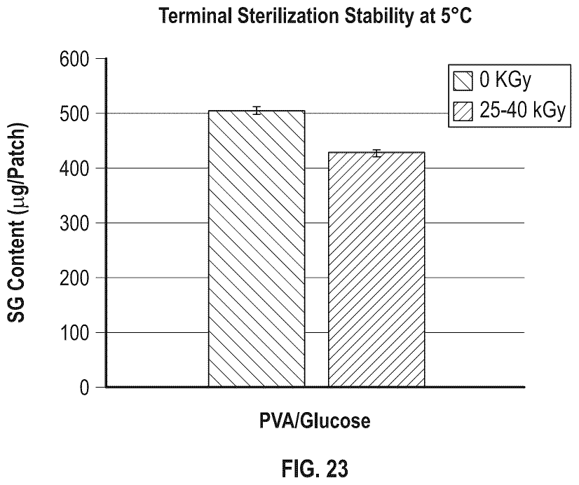

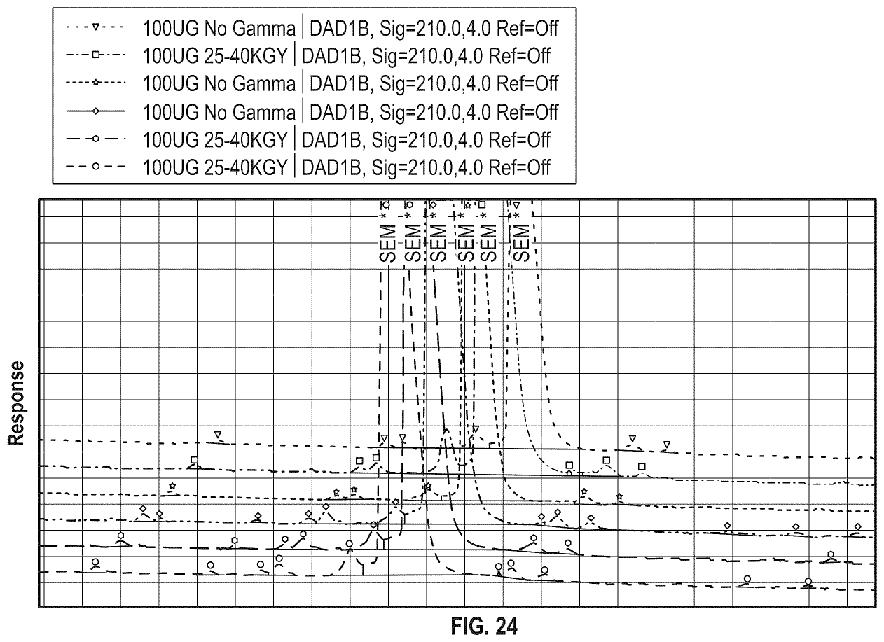

To continue to characterize formulations for gamma irradiation compatibility, higher dose SG-dMPs with a polycaprolactone (PCL) base were prepared and irradiated. These SG-dMPs contained 500 mcg of semaglutide and 3000 mcg of excipients with a 70:30 PVA: glucose excipient composition, and a 6:1 excipient to active ratio. After 2.5 months of storage at 5° C., patches exposed to 25-40 kGy of gamma radiation retained 85% of the SG content observed in the non-irradiated samples (FIG. 23). The appearance of several new minor peaks can be observed in the chromatograms of the irradiated samples, when compared to the unirradiated controls (FIG. 24).

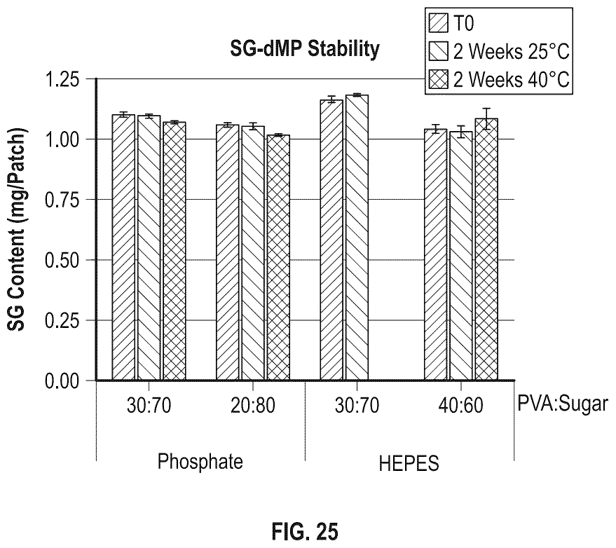

To continue to optimize higher dose SG-dMPs, the lead PVA/glucose excipient composition was used to manufacture microneedle arrays with a 1:1 excipient to active ratio and a target dose of 1.25 mg of SG. Different PVA: glucose ratios were evaluated, and formulations were prepared in 0.1 M phosphate buffer (pH 7) and in 50 mM HEPES buffer with 150 mM sodium chloride and 5 mM calcium chloride (pH 7.3) (FIG. 25). A photocurable medical grade adhesive was used to form the base of the microneedle array.

### EMBODIMENTS

Embodiment 1. A microneedle comprising: a microneedle body comprising a tip end portion configured for insertion into a biological tissue, wherein at least the tip end portion comprises: a mixture of a GLP-1 agonist and an excipient composition which comprises a carbohydrate and a polymer, wherein the mass ratio of the carbohydrate to the polymer in the excipient composition is at least 1.5:1.

Embodiment 2. The microneedle of Embodiment 1, wherein the carbohydrate is a sugar and the polymer is a hydrophilic polymer.

Embodiment 3. The microneedle of Embodiment 1 or 2, wherein the mass ratio of the carbohydrate to the polymer in the excipient composition is between 1.5:1 and 8:1.

Embodiment 4. The microneedle of any one of Embodiments 1 to 3, wherein the carbohydrate is selected from sucrose, glucose, dextrose, or a combination thereof.

Embodiment 5. The microneedle of any one of Embodiments 1 to 4, wherein the polymer is selected from a polyvinyl alcohol (PVA), a polyvinyl pyrrolidone (PVP), a polyethylene glycol (PEG), or a combination thereof.

Embodiment 6. The microneedle of Embodiment 5, wherein the polymer is PVA having a molecular weight between 5,000 and 60,000 Daltons.

Embodiment 7. The microneedle of Embodiment 5, wherein the polymer is PVP having a molecular weight between 5,000 and 60,000 Daltons.

Embodiment 8. The microneedle of Embodiment 5, wherein the polymer is PEG having a molecular weight between 5,000 and 60,000 Daltons.

Embodiment 9. The microneedle of any one of Embodiments 1 to 8, wherein the GLP-1 agonist is selected from semaglutide, liraglutide, dulaglutide, exenatide, tirzepatide, or a combination thereof.

Embodiment 10. The microneedle of any one of Embodiments 1 to 9, wherein the mass ratio of the GLP-1 agonist to the excipient composition in the mixture is between 0.5:1 and 2:1.

Embodiment 11. The microneedle of any one of Embodiments 1 to 10, wherein the mass ratio of the GLP-1 agonist to the excipient composition in the mixture is about 1:1.

Embodiment 12. The microneedle of any one of Embodiments 1 to 11, wherein the GLP-1 agonist comprises semaglutide, the polymer comprises PVA, and the carbohydrate comprises dextrose.

Embodiment 13. The microneedle of Embodiment 12, wherein the mass ratio of dextrose to PVA in the excipient composition is between 1.5:1 and 4.5:1, preferably 4:1.

Embodiment 14. The microneedle of any one of Embodiments 1 to 13, wherein the excipient composition comprises PVA, dextrose, and phosphate buffer.

Embodiment 15. The microneedle of any one of Embodiments 1 to 11, wherein the excipient composition comprises 60% by weight dextrose and 40% by weight PVA.

Embodiment 16. The microneedle of any one of Embodiments 1 to 11, wherein the excipient composition comprises 70% by weight dextrose and 30% by weight PVA.

Embodiment 17. The microneedle of any one of Embodiments 1 to 11, wherein the excipient composition comprises 80% by weight dextrose and 20% by weight PVA.

Embodiment 18. The microneedle of any one of Embodiments 1 to 11, wherein the excipient composition comprises PVA and maltose.

Embodiment 19. The microneedle of any one of Embodiments 1 to 11, wherein the excipient composition comprises PVP, dextrose, and glycerol.

Embodiment 20. The microneedle of any one of Embodiments 1 to 11, wherein the excipient composition comprises PVA and glucose.

Embodiment 21. The microneedle of any one of Embodiments 1 to 11, wherein the excipient composition comprises 70% by weight glucose and 30% by weight PVA.

Embodiment 22. The microneedle of any one of Embodiments 1 to 21, in which the GLP-1 agonist has a thermostability of at least two weeks at 40° C.

Embodiment 23. A microneedle patch comprising: a base; and an array of the microneedles of any one of Embodiments 1 to 22 extending from the base.

Embodiment 24. The microneedle patch of Embodiment 23, wherein the array comprises at least 0.05 mg of the GLP-1 agonist or wherein the array comprises from 0.25 mg to 2.5 mg of the GLP-1 agonist per cm2 of the array.

Embodiment 25. The microneedle patch of Embodiment 23 or 24, wherein the array comprises from 0.25 mg to 5.0 mg of the GLP-1 agonist.

Embodiment 26. The microneedle patch of any one of Embodiments 23 to 25, wherein the tip end portions of the microneedles have a conical shape and a length from 500 to 900 microns.

Embodiment 27. The microneedle patch of any one of Embodiments 23 to 26, wherein the array has between 20 and 500 of the microneedles.

Embodiment 28. The microneedle patch of any one of Embodiments 23 to 27, wherein the microneedles of the array are positioned to define a substantially circular perimeter of the array viewed in an axial direction of the microneedles, the outer boundary having a diameter from about 0.5 cm to 2 cm.

Embodiment 29. The microneedle patch of Embodiment 28, wherein the perimeter of the array has a diameter of 0.5 cm and the array of microneedles comprises about 0.05 mg of the GLP-1 agonist.

Embodiment 30. The microneedle patch of Embodiment 28, wherein the perimeter of the array has a diameter of 1 cm and the array of microneedles comprises about 1.25 mg of the GLP-1 agonist.

Embodiment 31. The microneedle patch of any one of Embodiments 23 to 30, wherein the microneedles further comprise at least one funnel portion extending between the base and the tip portions.

Embodiment 32. The microneedle patch of Embodiment 31, wherein at least the tip portions of the microneedles are dissolvable and the at least one funnel portion is non-dissolvable in contact with interstitial fluid.

Embodiment 33. The microneedle patch of any one of Embodiments 23 to 32, which has been terminally sterilized.

Embodiment 34. A kit of parts comprising: a plurality of microneedle patches of any one of Embodiments 23 to 33; and instructions for using the microneedle patches to treat a subject, wherein the instructions comprise dosing frequency and/or directions for applying the microneedles of the microneedles patches to the subject's skin or other biological tissue.

Embodiment 35. The kit of Embodiment 34, wherein at least one microneedle patch of the plurality of microneedle patches contains a different dose of the GLP-1 agonist than another microneedle patch of the plurality of microneedle patches.

Embodiment 36. The kit of Embodiment 34 or 35, wherein the instructions comprise dose escalating and/or dose de-escalating instructions.

Embodiment 37. The kit of any one of Embodiments 34 to 36, wherein one or more of the plurality of patches each contains a dose from 0.1 to 1.5 mg of the GLP-1 agonist.

Embodiment 38. The kit of Embodiment 37, wherein one or more of the plurality of patches each contains a dose from 0.1 to 0.5 mg of the GLP-1 agonist.

Embodiment 39. A method for administering a GLP-1 agonist to a subject comprising: applying to a tissue of the subject at least one of the microneedle patches of any one of Embodiments 23 to 38, in a manner to cause the tip portions of the microneedles to be inserted into the tissue and to thereby release the GLP-1 agonist into the tissue.

Embodiment 40. The method of Embodiment 39, wherein the applying is effective to deliver at least 0.05 milligrams of the GLP-1 agonist from the at least one microneedle patch.

Embodiment 41. The method of Embodiment 39 or 40, wherein a delivery efficiency of the GLP-1 agonist from the at least one microneedle patch is at least 60%.

Embodiment 42. The method of Embodiment 41, wherein the delivery efficiency is at least 75%.

Embodiment 43. The method of Embodiment 41, wherein the delivery efficiency is from 85% to 95%.

Embodiment 44. The method of any one of Embodiments 39 to 43, wherein the applying is effective to administer a pharmaceutically acceptable amount of the GLP-1 agonist to the subject to treat diabetes, obesity, or another metabolic condition, or a non-metabolic condition, in the subject.

Embodiment 45. The method of any one of Embodiments 39 to 43, wherein the applying is effective to administer a pharmaceutically acceptable amount of the GLP-1 agonist to the subject to aid in weight loss in the subject or to treat an addiction, kidney disease, and/or cardiovascular disease in the subject.

Embodiment 46. The method of any one of Embodiments 39 to 45, which comprises weekly applying 1, 2, 3, 4, 5, 6, 7, or more of the microneedle patches to the subject.

That which is claimed is:

1. A microneedle comprising:
a microneedle body comprising a tip end portion configured for insertion into a biological tissue, wherein at least the tip end portion comprises:
a mixture of a GLP-1 agonist and an excipient composition which comprises a carbohydrate and a polymer, wherein the mass ratio of the carbohydrate to the polymer in the excipient composition is at least 1.5:1.

2. The microneedle of claim 1, wherein the carbohydrate is a sugar and the polymer is a hydrophilic polymer.

3. The microneedle of claim 1, wherein the mass ratio of the carbohydrate to the polymer in the excipient composition is between 1.5:1 and 8:1.

4. The microneedle of claim 1, wherein:
the carbohydrate is selected from sucrose, glucose, dextrose, or a combination thereof; and/or
the polymer is selected from a polyvinyl alcohol (PVA), a polyvinyl pyrrolidone (PVP), a polyethylene glycol (PEG), or a combination thereof.

5. The microneedle of claim 4, wherein the polymer has a molecular weight between 5,000 and 60,000 Daltons.

6. The microneedle of claim 1, wherein the GLP-1 agonist is selected from semaglutide, liraglutide, dulaglutide, exenatide, tirzepatide, or a combination thereof.

7. The microneedle of claim 1, wherein the mass ratio of the GLP-1 agonist to the excipient composition in the mixture is between 0.5:1 and 2:1, or is about 1:1.

8. The microneedle of claim 1, wherein the GLP-1 agonist comprises semaglutide, the polymer comprises PVA, and the carbohydrate comprises dextrose.

9. The microneedle of claim 8, wherein the mass ratio of dextrose to PVA in the excipient composition is between 1.5:1 and 4.5:1, preferably 4:1.

10. The microneedle of claim 1, wherein the excipient composition comprises PVA, dextrose, and phosphate buffer.

11. The microneedle of claim 1, wherein the excipient composition comprises:
60% by weight dextrose and 40% by weight PVA;
70% by weight dextrose and 30% by weight PVA; or
80% by weight dextrose and 20% by weight PVA.

12. The microneedle of claim 1, wherein the excipient composition comprises PVA and maltose.

13. The microneedle of claim 1, wherein the excipient composition comprises PVP, dextrose, and glycerol.

14. The microneedle of claim 1, wherein the excipient composition comprises PVA and glucose, optionally wherein the excipient composition comprises 70% by weight glucose and 30% by weight PVA.

15. The microneedle of claim 1, in which the GLP-1 agonist has a thermostability of at least two weeks at 40° C.

16. A microneedle patch comprising:
a base; and
an array of the microneedles of claim 1 extending from the base.

17. The microneedle patch of claim 16, wherein the array of the microneedles comprises at least 0.05 mg of the GLP-1 agonist, optionally from 0.25 mg to 5.0 mg of the GLP-1 agonist.

18. The microneedle patch of claim 16, wherein the array comprises from 0.25 mg to 2.5 mg of the GLP-1 agonist per cm² of the array.

19. The microneedle patch of claim 16, wherein the tip end portions of the microneedles have a conical shape and a length from 500 to 900 microns.

20. The microneedle patch of claim 16, wherein the array has between 20 and 500 of the microneedles.

21. The microneedle patch of claim 16, wherein the microneedles of the array are positioned to define a substantially circular perimeter of the array viewed in an axial direction of the microneedles, the outer boundary having a diameter from about 0.5 cm to 2 cm.

22. The microneedle patch of claim 21, wherein:
the perimeter of the array has a diameter of 0.5 cm and the array of microneedles comprises about 0.05 mg of the GLP-1 agonist, or the perimeter of the array has a diameter of 1 cm and the array of microneedles comprises about 1.25 mg of the GLP-1 agonist.

23. The microneedle patch of claim 16, wherein the microneedles further comprise at least one funnel portion extending between the base and the tip portions.

24. The microneedle patch of claim 23, wherein at least the tip portions of the microneedles are dissolvable and the at least one funnel portion is non-dissolvable in contact with interstitial fluid.

25. The microneedle patch of claim 16, which has been terminally sterilized.

26. A kit of parts comprising:
a plurality of microneedle patches of claim 16; and
instructions for using the microneedle patches to treat a subject, wherein the instructions comprise dosing frequency and/or directions for applying the microneedles of the microneedles patches to the subject's skin or other biological tissue.

27. The kit of claim 26, wherein at least one microneedle patch of the plurality of microneedle patches contains a different dose of the GLP-1 agonist than another microneedle patch of the plurality of microneedle patches.

28. The kit of claim 26, wherein the instructions comprise dose escalating and/or dose de-escalating instructions.

29. The kit of claim 26, wherein one or more of the plurality of patches each contains a dose from 0.1 to 1.5 mg of the GLP-1 agonist, or a dose from 0.1 to 0.5 mg of the GLP-1 agonist.

30. A method for administering a GLP-1 agonist to a subject comprising:
applying to a tissue of the subject at least one of the microneedle patches of claim 16 in a manner to cause the tip portions of the microneedles to be inserted into the tissue and to thereby release the GLP-1 agonist into the tissue.

31. The method of claim 30, wherein the applying is effective to deliver at least 0.05 milligrams of the GLP-1 agonist from the at least one microneedle patch.

32. The method of claim 30, wherein a delivery efficiency of the GLP-1 agonist from the at least one microneedle patch is at least 60%, preferably at least 75%.

33. The method of claim 32, wherein the delivery efficiency is from 85% to 95%.

34. The method of claim 30, wherein the applying is effective to administer a pharmaceutically acceptable amount of the GLP-1 agonist to the subject to treat diabetes, obesity, or another metabolic condition, or a non-metabolic condition, in the subject.

35. The method of claim 30, wherein the applying is effective to administer a pharmaceutically acceptable amount of the GLP-1 agonist to the subject to aid in weight loss in the subject or to treat an addiction, kidney disease, and/or cardiovascular disease in the subject.

36. The method of claim 30, which comprises weekly applying 1, 2, 3, 4, 5, 6, 7, or more of the microneedle patches to the subject.
